(19) United States
(12) Patent Application Publication
(10) Pub. No.: US 2021/0355222 A1
(43) Pub. Date: Nov. 18, 2021

(54) Methods of Treating Cancer

(71) Applicant: Jounce Therapeutics, Inc., Cambridge, MA (US)

(72) Inventors: Lara Lewis McGrath, Arlington, MA (US); Christopher Harvey, Boston, MA (US); Deborah Law, Cambridge, MA (US); Amanda Hanson, Cambridge, MA (US)

(73) Assignee: Jounce Therapeutics, Inc., Cambridge, MA (US)

(21) Appl. No.: 17/286,104

(22) PCT Filed: Oct. 25, 2019
(86) PCT No.: PCT/US2019/058027

§ 371 Date: Apr. 16, 2021

Related U.S. Application Data
(60) Provisional application No. 62/751,433, filed on Oct. 26, 2018.

(51) Int. Cl. C07K 16/28; A61K 45/06; G01N 33/50; G01N 33/68; A61P 35/00

(52) U.S. Cl. CPC C07K 16/2818; A61K 45/06; C07K 16/2827; A61K 2039/545; G01N 33/6893; A61P 35/00; G01N 33/5044

(57) ABSTRACT

The present disclosure provides methods of treating cancer and methods for selecting treatment approaches for cancer.

### CROSS-REFERENCE TO RELATED APPLICATIONS

This application claims the benefit of priority of U.S. Provisional Application No. 62/751,433, filed Oct. 26, 2018, which is incorporated by reference herein in its entirety for any purpose.

### FIELD

The present disclosure relates to methods of treating cancer and methods for selecting treatment approaches for cancer.

### BACKGROUND

ICOS is a member of the B7/CD28/CTLA-4 immunoglobulin superfamily and is specifically expressed on T cells. Unlike CD28, which is constitutively expressed on T cells and provides co-stimulatory signals necessary for full activation of resting T cells, ICOS is expressed only after initial T cell activation.

ICOS has been implicated in diverse aspects of T cell responses (reviewed in Simpson et al., Curr. Opin. Immunol., 22: 326-332, 2010). It plays a role in the formation of germinal centers, T/B cell collaboration, and immunoglobulin class switching. ICOS-deficient mice show impaired germinal center formation and have decreased production of interleukin IL-10. These defects have been specifically linked to deficiencies in T follicular helper cells. ICOS also plays a role in the development and function of other T cell subsets, including Th1, Th2, and Th17. Notably, ICOS co-stimulates T cell proliferation and cytokine secretion associated with both Th1 and Th2 cells. Accordingly, ICOS knock-out mice demonstrate impaired development of autoimmune phenotypes in a variety of disease models, including diabetes (Th1), airway inflammation (Th2), and EAE neuro-inflammatory models (Th17).

In addition to its role in modulating T effector (Teff) cell function, ICOS also modulates T regulatory cells (Tregs). ICOS is expressed at high levels on Tregs, and has been implicated in Treg homeostasis and function.

Upon activation, ICOS, a disulfide-linked homodimer, induces a signal through the PI3K and AKT pathways. Subsequent signaling events result in expression of lineage specific transcription factors (e.g., T-bet, GATA-3) and, in turn, effects on T cell proliferation and survival.

ICOS ligand (ICOSL; B7-H2; B7RP1; CD275; GL50), also a member of the B7 superfamily, is the only ligand for ICOS and is expressed on the cell surfaces of B cells, macrophages, and dendritic cells. ICOSL functions as a non-covalently linked homodimer on the cell surface in its interaction with ICOS. Human ICOSL, although not mouse ICOSL, has been reported to bind to human CD28 and CTLA-4 (Yao et al., Immunity, 34: 729-740, 2011).

### SUMMARY

The present disclosure provides methods of treating cancer in a subject (e.g., a human patient) in need thereof, the methods including, but not limited to, administering an effective amount of anti-ICOS agonist antibody to the subject, wherein the subject exhibits a low peripheral T-cell receptor (TCR) clonality, or a peripheral T-cell receptor (TCR) clonality of 0.50 or less. In various embodiments, the method of treating cancer in a subject in need thereof comprises (i) detecting that the subject exhibits a peripheral T-cell receptor (TCR) clonality of 0.50 or less, and (ii) following step (i), administering an effective amount of anti-ICOS agonist antibody to the subject.

In various embodiments, the method of treating cancer in a subject in need thereof comprises (i) determining whether the subject exhibits a low peripheral T-cell receptor (TCR) clonality, or a peripheral T-cell receptor (TCR) clonality of 0.50 or less, and (ii) following step (i), if the subject exhibits the peripheral TCR clonality of 0.50 or less, administering an effective amount of anti-ICOS agonist antibody to the subject.

In various embodiments, the method of treating cancer in a subject in need thereof comprises (i) measuring the level of peripheral T-cell receptor (TCR) clonality, and (ii) following step (i), if the subject has a low peripheral T-cell receptor (TCR) clonality, or a peripheral TCR clonality of 0.50 or less, administering an effective amount of anti-ICOS agonist antibody to the subject.

In some embodiments, (ii) administering an effective amount of anti-ICOS agonist antibody to the subject is performed when the subject is determined to exhibit a peripheral TCR clonality of 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

In various embodiments, the method of treating cancer in a subject in need thereof comprises (i) providing a subject having cancer previously determined to have a low peripheral T-cell receptor (TCR) clonality, or a peripheral T-cell receptor (TCR) clonality of 0.50 or less, and (ii) administering an effective amount of anti-ICOS agonist antibody to the subject. In some embodiments, the subject is previously determined to have peripheral TCR clonality of 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

In various embodiments, the method of treating cancer in a subject in need thereof comprises administering an effective amount of anti-ICOS agonist antibody to the subject, wherein the subject exhibits a low peripheral T-cell receptor (TCR) clonality, or a peripheral T-cell receptor (TCR) clonality of 0.50 or less. In some embodiments, the subject exhibits a peripheral TCR clonality of 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

In some embodiments, the peripheral TCR clonality is 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

In some embodiments, detecting that the cancer exhibits a low peripheral T-cell receptor (TCR) clonality, or a peripheral TCR clonality of 0.50 or less or determining whether the cancer exhibits a low peripheral T-cell receptor (TCR) clonality, or a peripheral TCR clonality of 0.50 or less comprises measuring the level of peripheral TCR clonality.

In some embodiments, measuring the level of peripheral TCR clonality comprises testing a sample from the subject.

In some embodiments, measuring the level of peripheral TCR clonality comprises isolating peripheral cells from a tumor or other appropriate tissue associated with said cancer in said subject and testing the cells for presence of a low peripheral T-cell receptor (TCR) clonality, or a peripheral TCR clonality of 0.5 or less.

In some embodiments, measuring the level of peripheral TCR clonality comprises isolating peripheral blood mononuclear cells (PBMC) from a tumor or other appropriate tissue associated with said cancer in said subject and testing the cells for presence of a low peripheral T-cell receptor (TCR) clonality, or a peripheral TCR clonality of 0.5 or less.

In some embodiments, measuring the level of peripheral TCR clonality comprises isolating nucleic acid from the peripheral blood of said subject and sequencing all or a portion of a region encoding T cell receptor protein. In some embodiments, the region of the T cell receptor is a beta chain. In some embodiments, the region of the T cell receptor is a variable region or a joining region. In some embodiments, the region of the T cell receptor is a variable region of a beta chain. In some embodiments, the subject is a human subject.

In some embodiments, the sample from the subject is peripheral blood mononuclear cells (PBMC).

In some embodiments, the method further comprising (iii) after the administration, obtaining one or more peripheral blood test samples from the subject, (iii) measuring ICOS levels of CD4+ T cells present in the one or more peripheral blood test samples, (iv) determining if there is a population of CD4+ T cells having elevated ICOS in any of the one or more peripheral blood test samples when compared to a control, wherein detection of increased ICOS relative to a control indicates that the subject may benefit from the administration.

In various embodiments, a method of treating cancer in a subject in need thereof comprises (i) detecting that the subject exhibits a low peripheral T-cell receptor (TCR) clonality, and (ii) following step (i), administering an effective amount of anti-ICOS agonist antibody to the subject.

In various embodiments, a method of treating cancer in a subject in need thereof comprises (i) determining whether the subject exhibits a low peripheral T-cell receptor (TCR) clonality, and (ii) following step (i), if the subject exhibits a low peripheral TCR clonality, administering an effective amount of anti-ICOS agonist antibody to the subject.

In various embodiments, a method of treating cancer in a subject in need thereof comprises (i) measuring the level of a low peripheral T-cell receptor (TCR) clonality, and (ii) following step (i), if the subject has a low peripheral TCR clonality, administering an effective amount of anti-ICOS agonist antibody to the subject.

In various embodiments, a method of treating cancer in a subject in need thereof comprises (i) providing a subject having cancer previously determined to have a low peripheral T-cell receptor (TCR) clonality, and (ii) administering an effective amount of anti-ICOS agonist antibody to the subject.

In various embodiments, a method of treating cancer in a subject in need thereof comprises administering an effective amount of anti-ICOS agonist antibody to the subject, wherein the subject exhibits a low peripheral T-cell receptor (TCR) clonality.

In various embodiments, the anti-ICOS agonist antibody is chosen from JTX-2011, BMS-986226, and GSK3359609. In some embodiment, the anti-ICOS agonist antibody is JTX-2011.

In various embodiments, the anti-ICOS agonist antibody comprises a heavy chain and a light chain, and further comprises at least one CDR selected from: (a) an HCDR1 comprising the amino acid sequence of SEQ ID NO: 5; (b) an HCDR2 comprising the amino acid sequence of SEQ ID NO: 6; (c) an HCDR3 comprising the amino acid sequence of SEQ ID NO: 7; (d) an LCDR1 comprising the amino acid sequence of SEQ ID NO: 8; (e) an LCDR2 comprising the amino acid sequence of SEQ ID NO: 9; and (f) an LCDR3 comprising the amino acid sequence of SEQ ID NO: 10, wherein one or more of the CDRs comprises 1 or 2 amino acid substitutions. In some embodiments, the anti-ICOS agonist antibody comprises (a) an HCDR1 comprising the amino acid sequence of SEQ ID NO: 5; (b) an HCDR2 comprising the amino acid sequence of SEQ ID NO: 6; (c) an HCDR3 comprising the amino acid sequence of SEQ ID NO: 7; (d) an LCDR1 comprising the amino acid sequence of SEQ ID NO: 8; (e) an LCDR2 comprising the amino acid sequence of SEQ ID NO: 9; and (0 an LCDR3 comprising the amino acid sequence of SEQ ID NO: 10. In some embodiments, the anti-ICOS agonist antibody comprises (a) an HCDR1 comprising the amino acid sequence of SEQ ID NO: 5; (b) an HCDR2 comprising the amino acid sequence of SEQ ID NO: 6; (c) an HCDR3 comprising the amino acid sequence of SEQ ID NO: 7; (d) an LCDR1 comprising the amino acid sequence of SEQ ID NO: 8; (e) an LCDR2 comprising the amino acid sequence of SEQ ID NO: 9; and (f) an LCDR3 comprising the amino acid sequence of SEQ ID NO: 10.

In various embodiments, the anti-ICOS agonist antibody comprises (a) a heavy chain variable domain (VH) sequence having at least 90%, 91%, 92%, 93%, 94%, 95%, 96%, 97%, 98%, 99%, or 100% sequence identity to the amino acid sequence of SEQ ID NO: 3; and/or (b) a light chain variable domain (VL) having at least 90%, 91%, 92%, 93%, 94%, 95%, 96%, 97%, 98%, 99%, or 100% sequence identity to the amino acid sequence of SEQ ID NO: 4. In some embodiments, the anti-ICOS agonist antibody comprises (a) a heavy chain variable domain (VH) sequence comprising the amino acid sequence of SEQ ID NO: 3, and (b) a light chain variable domain (VL) comprising the amino acid sequence of SEQ ID NO: 4.

In various embodiments, the anti-ICOS agonist antibody comprises (a) a heavy chain comprising the amino acid sequence of SEQ ID NO: 1 and/or (b) a light chain comprising the amino acid sequence of SEQ ID NO: 2. In some embodiments, the anti-ICOS agonist antibody comprises (a) a heavy chain comprising the amino acid sequence of SEQ ID NO: 1 and (b) a light chain comprising the amino acid sequence of SEQ ID NO: 2.

In various embodiments, the anti-ICOS agonist antibody is administered at a dosage of from 0.1 mg/kg to 0.3 mg/kg. In some embodiments, the anti-ICOS agonist antibody is administered at a dosage of 0.1 mg/kg, 0.2 mg/kg, or 0.3 mg/kg. In some embodiments, the anti-ICOS agonist antibody is administered at a dosage of 0.3 mg/kg.

In various embodiments, the anti-ICOS agonist antibody is administered at a frequency of weekly, once every two weeks, once every three weeks, once every four weeks, once every six weeks, once every nine weeks, or once every twelve weeks.

In some embodiments, the method further includes administering an additional therapeutic agent with the anti-ICOS agonist antibody. In some embodiments, the additional therapeutic agent is an immunotherapeutic agent. In some embodiments, the additional therapeutic agent is at least one of (i) an anti-CTLA-4 antagonist antibody, (ii) an anti-PD-1 or anti-PD-L1 antagonist antibody, and (iii) an agent listed in Table 2.

In various embodiments, the additional therapeutic agent includes an anti-CTLA-4 antagonist antibody. In some embodiments, the anti-CTLA-4 antagonist antibody is selected from ipilimumab, tremelimumab, and BMS-986249. In some embodiments, the anti-CTLA-4 antagonist antibody is ipilimumab.

In various embodiments, the additional therapeutic agent includes an anti-PD-1 or anti-PD-L1 antagonist antibody. In some embodiments, the anti-PD-1 or anti-PD-L1 antagonist antibody is chosen from avelumab, atezolizumab, CX-072, pembrolizumab, nivolumab, cemiplimab, spartalizumab, tislelizumab, JNJ-63723283, genolimzumab, AMP-514, AGEN2034, durvalumab, and JNC-1. In some embodiments, the anti-PD-1 or anti-PD-L1 antagonist antibody is chosen from pembrolizumab, nivolumab, atezolizumab, avelumab, and duravalumab.

In some embodiments, the additional therapeutic agent includes one or more of the agents listed in Table 2.

In some embodiments, the additional therapeutic agent further includes a chemotherapy agent. In some embodiments, the chemotherapy agent is selected from one or more of capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, pemetrexed, vinorelbine, vincristine, erlotinib, afatinib, gefitinib, crizotinib, dabrafenib, trametinib, vemurafenib, and cobimetanib.

In some embodiments, the method further includes administering radiation therapy.

In various embodiments, the additional therapeutic agent is administered every week, every two weeks, every three weeks, every four weeks, every six weeks, every nine weeks, and every twelve weeks.

In various embodiments, the cancer is selected from gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, and head and neck squamous cell cancer (HNSCC). In some embodiments, the cancer is gastric cancer. In some embodiments, the cancer is non-small cell lung cancer. In some embodiments, the cancer is metastasized to the ovary of said subject (i.e., a Krukenberg tumor).

Other features and advantages of the present disclosure will be apparent from the following detailed description, the drawings, and the claims.

### BRIEF DESCRIPTION OF THE DRAWINGS

FIG. 1 shows comparison of clonality in TCR repertoire in peripheral cells in cancer patients prior to receiving therapeutic anti-ICOS antibody (JTX-2011) monotherapy between populations having an emergence of an ICOS^{hi}CD4+ T cell population (Y) and populations with no emergence of an ICOS^{hi} CD4+ T cell population (N). The y-axis represents TCR clonality levels. The middle line of the box mark is the median for each group, the edges of the box mark are the 25^{th} and 75^{th} percentiles, and the dark dots are outliers. The ICOS^{hi} CD4+ T cell population was detected by flow cytometry.

FIG. 2 shows a waterfall plot comparing the percent change from baseline in target lesion with the emergence of an ICOS^{hi} CD4+ T cell population in cancer patients receiving a JTX-2011 monotherapy or a combination therapy of JTX-2011 and nivolumab. For each patient (each bar), the percent change in measurable tumor at best response is displayed by an emergence or no emergence of an ICOS^{hi} CD4+ T cell population.

### DETAILED DESCRIPTION

In general, the present disclosure is based the discovery that patients having low TCR clonality (i.e., high diversity) are enriched among patients that have shown a response to treatment in humans with an anti-ICOS agonist and, optionally, a PD-1 antagonist. Accordingly, methods of treating cancer according to TCR clonality status are provided. The methods include administering an effective amount of anti-ICOS agonist antibody to a subject, wherein the subject has a low TCR clonality. Also provided are methods for treating cancer including detecting that the cancer exhibits a low TCR clonality, and administering an effective amount of anti-ICOS agonist antibody to the subject. Also, other anti-cancer therapies listed herein, e.g., using immunotherapeutic agents, can be used in combination with the treatment with the anti-ICOS agonist antibody.

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

### I. Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that some embodiments of the present disclosure described herein may include “consisting” and/or “consisting essentially of” embodiments. In the claims and disclosure herein, terms such as “having” or “including” are to be construed equivalently to the term “comprising.”

As used herein, the singular form “a,” “an,” and “the” includes plural references unless indicated otherwise.

Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

All numbers herein are approximate and are intended to account for both normal measurement errors as well as rounding to the nearest significant figure.

The terms “nucleic acid molecule,” “nucleic acid,” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

“ICOS” and “inducible T-cell costimulatory” as used herein refer to any native ICOS that results from expression and processing of ICOS in a cell. The term includes ICOS from any vertebrate source, including mammals such as primates (e.g., humans and cynomolgus monkeys) and rodents (e.g., mice and rats), unless otherwise indicated. The term also includes naturally occurring variants of ICOS, e.g., splice variants or allelic variants. The amino acid sequence of an exemplary human ICOS precursor protein, with signal sequence (amino acids 1-20) is shown in SEQ ID NO: 11. The amino acid sequence of an exemplary mature human ICOS is shown in SEQ ID NO: 12. The intracellular portion of ICOS is indicated in Table 3 by underlining within SEQ ID NOs: 11 and 12. The amino acid sequence of an exemplary mouse ICOS precursor protein, with signal sequence (amino acids 1-20) is shown in SEQ ID NO: 13. The amino acid sequence of an exemplary mature mouse ICOS is shown in SEQ ID NO: 14. The amino acid sequence of an exemplary rat ICOS precursor protein, with signal sequence (amino acids 1-20) is shown in SEQ ID NO: 15. The amino acid sequence of an exemplary mature rat ICOS is shown in SEQ ID NO: 16. The amino acid sequence of an exemplary cynomolgus monkey ICOS precursor protein, with signal sequence (amino acids 1-20) is shown in SEQ ID NO: 17. The amino acid sequence of an exemplary mature cynomolgus monkey ICOS is shown in SEQ ID NO: 18.

The term “specifically binds” to an antigen or epitope is a term that is well-understood in the art, and methods to determine such specific binding are also well known in the art. A molecule is said to exhibit “specific binding” or “preferential binding” if it reacts or associates more frequently, more rapidly, with greater duration, and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an ICOS epitope is an antibody that binds this epitope with greater affinity, avidity, more readily, and/or with greater duration than it binds to other ICOS epitopes or non-ICOS epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, “substantially pure” refers to material which is at least 50% pure (that is, free from contaminants), more preferably, at least 90% pure, more preferably, at least 95% pure, yet more preferably, at least 98% pure, and most preferably, at least 99% pure.

As used herein, the term “epitope” refers to a site on a target molecule (for example, an antigen, such as a protein, nucleic acid, carbohydrate, or lipid) to which an antigen-binding molecule (for example, an antibody, antibody fragment, or scaffold protein containing antibody binding regions) binds. Epitopes often include a chemically active surface grouping of molecules such as amino acids, polypeptides, or sugar side chains and have specific three-dimensional structural characteristics as well as specific charge characteristics. Epitopes can be formed both from contiguous and/or juxtaposed noncontiguous residues (for example, amino acids, nucleotides, sugars, or lipid moieties) of the target molecule. Epitopes formed from contiguous residues, also called linear epitopes (for example, amino acids, nucleotides, sugars, or lipid moieties), typically are retained on exposure to denaturing solvents whereas epitopes formed from non-contiguous residues, also called non-linear or conformational epitopes, are formed by tertiary folding, and typically are lost on treatment with denaturing solvents. An epitope may include, but is not limited to, at least 3, at least 5, or 8-10 residues (for example, amino acids or nucleotides). In some examples, an epitope is less than 20 residues (for example, amino acids or nucleotides) in length, less than 15 residues, or less than 12 residues.

Two antibodies may bind to the same epitope within an antigen, or to overlapping epitopes, if they exhibit competitive binding for the antigen. Accordingly, in some embodiments, an antibody is said to “cross-compete” with another antibody if it specifically interferes with the binding of the antibody to the same or an overlapping epitope.

The term “antibody” herein is used in the broadest sense and encompasses various antibody structures, including but not limited to, monoclonal antibodies, polyclonal antibodies, multispecific antibodies (for example, bispecific (such as Bi-specific T-cell engagers) and trispecific antibodies), and antibody fragments as long as they exhibit a desired antigen-binding activity.

The term antibody includes, but is not limited to, fragments that are capable of binding to an antigen, such as Fv, single-chain Fv (scFv), Fab, Fab′, di-scFv, sdAb (single domain antibody), and (Fab′)2 (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, and/or a combination of the Kabat, Chothia, AbM, and/or contact definitions. Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991)). The AbM definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, H26-H35B of H1, 50-58 of H2, and 95-102 of H3. The Contact definition can include, for example, CDRs (CDR-L1, CDR-L2, CDR-L3, CDR-H1, CDR-H2, and CDR-H3) at amino acid residues 30-36 of L1, 46-55 of L2, 89-96 of L3, 30-35 of H1, 47-58 of H2, and 93-101 of H3. The Chothia definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, 26-32 . . . 34 of H1, 52-56 of H2, and 95-102 of H3. With the exception of CDR1 in VH, CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hyper variable region,” including hypervariable loops. Exemplary hypervariable loops occur at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)).

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, CH1, CH2, and CH3. Of course, non-function-altering deletions and alterations within the domains are encompassed within the scope of the term “heavy chain constant region,” unless designated otherwise. Non-limiting exemplary heavy chain constant regions include γ, δ, and α. Non-limiting exemplary heavy chain constant regions also include ϵ and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ϵ constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCR1, framework (FR) 2, LCD2, FR3, and LCD3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Non-limiting exemplary light chain constant regions include λ and K. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework derived from a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (KD). Affinity can be measured by common methods known in the art (such as, for example, ELISA KD, KinExA, bio-layer interferometry (BLI), and/or surface plasmon resonance devices (such as a

BIAcore® device), including those described herein).

The term “KD,” “Kd,” “Kd,” or “Kd value” as used herein, refers to the equilibrium dissociation constant of an antibody-antigen interaction.

The term “biological activity” refers to any one or more biological properties of a molecule (whether present naturally as found in vivo, or provided or enabled by recombinant means).

Biological properties include, but are not limited to, binding a receptor, inducing cell proliferation, inhibiting cell growth, inducing other cytokines, inducing apoptosis, and enzymatic activity. In some embodiments, biological activity of an ICOS protein includes, for example, costimulation of T cell proliferation and cytokine secretion associated with Th1 and Th2 cells; modulation of Treg cells; effects on T cell differentiation including modulation of transcription factor gene expression; induction of signaling through P13K and AKT pathways; and mediating ADCC.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

The phrase “substantially increased,” as used herein, denotes a sufficiently high degree of increase between a numeric value and a reference numeric value such that one of skill in the art would consider the difference between the two values to be of statistical significance within the context of the biological characteristic measured by said values. In some embodiments, the substantially increased numeric values is increased by greater than about any one of 10%, 20%, 25%, 30%, 35%, 40%, 45%, 50%, 60%, 70%, 80%, 90%, or 100% compared to the reference value.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an

RNA polynucleotide. Thus, a DNA polynucleotide that is contained in a vector inside a host cell may be referred to as “isolated.”

The terms “individual,” “patient,” or “subject” are used interchangeably herein to refer to an animal, for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

The term “sample” or “patient sample” as used herein, refers to a composition that is obtained or derived from a subject of interest that contains a cellular and/or other molecular entity that is to be characterized and/or identified, for example, based on physical, biochemical, chemical, and/or physiological characteristics. For example, the phrase “test sample,” and variations thereof, refers to any sample obtained from a subject of interest that would be expected or is known to contain a cellular and/or molecular entity that is to be characterized. By “tissue or cell sample” is meant a collection of similar cells obtained from a tissue of a subject or patient. The source of the tissue or cell sample may be blood (e.g., peripheral blood) or any blood constituents; solid tissue as from a fresh, frozen, and/or preserved organ or tissue sample or biopsy or aspirate; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. In some embodiments, a sample includes peripheral blood obtained from a subject or patient, which includes CD4+ cells. In some embodiments, a sample includes CD4+ cells isolated from peripheral blood. In some embodiments, a sample includes peripheral cells, including peripheral blood mononuclear cells (PBMCs).

A “control,” “control sample,” “reference,” or “reference sample” as used herein, refers to any sample, standard, or level that is used for comparison purposes. A control or reference may be obtained from a healthy and/or non-diseased sample. In some examples, a control or reference may be obtained from an untreated sample or patient. In some examples, a reference is obtained from a non-diseased or non-treated sample of a subject individual. In some examples, a reference is obtained from one or more healthy individuals who are not the subject or patient. In some embodiments, a control sample, reference sample, reference cell, or reference tissue is obtained from the patient or subject at a time point prior to one or more administrations of a treatment (e.g., one or more anti-cancer treatments), or prior to being subjected to any of the methods of the present disclosure.

A “disease” or “disorder” as used herein refers to a condition where treatment is needed and/or desired. In some embodiments, the disease or disorder is cancer. “Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growth, such as dysplasia. Examples of cancer include but are not limited to, carcinoma, lymphoma, blastoma, sarcoma, and leukemia. More particular non-limiting examples of such cancers include gastric cancer, breast cancer (e.g., triple negative breast cancer (TNBC)), non-small cell lung cancer (NSCLC), squamous cell cancer, small-cell lung cancer, pituitary cancer, esophageal cancer, astrocytoma, soft tissue sarcoma, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, colon cancer, colorectal cancer, endometrial or uterine carcinoma (including uterine corpus endometrial carcinoma), salivary gland carcinoma, kidney cancer, renal cancer, liver cancer, prostate cancer, vulval cancer, thyroid cancer, hepatic carcinoma, brain cancer, testis cancer, cholangiocarcinoma, gallbladder carcinoma, melanoma, and various types of head and neck cancer. These cancers, and others, can be treated or analyzed according to the methods of the present disclosure.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example, metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder or a complete response to the therapy used.

“Ameliorating” means a lessening or improvement of one or more symptoms as compared to not administering an anti-cancer therapy. “Ameliorating” also includes shortening or reduction in duration of a symptom.

In the context of cancer, the term “treating” includes any or all of: inhibiting growth of cancer cells, inhibiting replication of cancer cells, lessening of overall tumor burden, and ameliorating one or more symptoms associated with the disease. “Preventing,” as used herein, includes providing prophylaxis with respect to the occurrence or recurrence of a disease in a subject that may be predisposed to the disease but has not yet been diagnosed with the disease. Unless otherwise specified, the terms “reduce,” “inhibit,” or “prevent” do not denote or require complete prevention over all time.

“Administering” refers to the physical introduction of a composition comprising a therapeutic agent to a subject, using any of the various methods and delivery systems known to those skilled in the art. Routes of administration for the anti-ICOS agonist antibody, for example, include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include a topical, epidermal or mucosal route of administration, for example, orally, intranasally, vaginally, rectally, sublingually or topically. Administering can also be performed, for example, once, a plurality of times, and/or over one or more extended periods.

The term “effective amount” or “therapeutically effective amount” refers to an amount of a drug effective to treat a disease or disorder in a subject. In certain embodiments, an effective amount refers to an amount effective, at dosages and for periods of time necessary, to achieve the desired therapeutic or prophylactic result. A therapeutically effective amount of an anti-ICOS agonist antibody of the invention may vary according to factors such as the disease state, age, sex, and weight of the individual, and the ability of the antibody or antibodies to elicit a desired response in the individual. A therapeutically effective amount encompasses an amount in which any toxic or detrimental effects of the antibody or antibodies are outweighed by the therapeutically beneficial effects. In some embodiments, the expression “effective amount” refers to an amount of the antibody that is effective for treating the cancer. A “therapeutic amount” refers to a dosage of a drug that has been approved for use by a regulatory agency. A “subtherapeutic amount” as used herein refers to a dosage of a drug or therapeutic agent that is significantly lower than the approved dosage. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

A “chemotherapeutic agent” is a chemical compound useful in the treatment of cancer. Examples of chemotherapeutic agents include, but are not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegal1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Ill.), and Taxotere® doxetaxel (Rhône-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylomithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

An “immunotherapy agent” includes, for example, an agent that may enhance the immune response of the patient to the cancer. Exemplary immunotherapy agents include anti-CTLA4 antagonist antibodies, anti-0X40 agonist antibodies, PD-1 therapies, TIGIT antagonists, IDO inhibitors, RORy agonists, certain cancer vaccines and other therapies described further below.

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (for example, severity of disease, progression/non-progression/improvement, etc.). While the present disclosure may provide exemplary predetermined levels, it is well-known that cutoff values may vary depending on the nature of the immunoassay (for example, antibodies employed, etc.). It further is well within the skill of one of ordinary skill in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce, or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time.

As used herein, “delaying development of a disease” means to defer, hinder, slow, retard, stabilize, suppress, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

A “therapeutically effective amount” of a substance/molecule, agonist, or antagonist may vary according to factors such as the disease state, age, sex, and weight of the individual, and the ability of the substance/molecule, agonist, or antagonist to elicit a desired response in the individual. A therapeutically effective amount is also one in which any toxic or detrimental effects of the substance/molecule, agonist, or antagonist are outweighed by the therapeutically beneficial effects. A therapeutically effective amount may be delivered in one or more administrations. A therapeutically effective amount refers to an amount effective, at dosages, and for periods of time necessary, to achieve the desired therapeutic and/or prophylactic result. The therapeutically effective amount of the treatment of the present disclosure can be measured by various endpoints commonly used in evaluating cancer treatments, including, but not limited to: extending survival (including OS and PFS); resulting in an objective response (including a CR or a PR); tumor regression, tumor weight or size shrinkage, longer time to disease progression, increased duration of survival, longer PFS, improved OS rate, increased duration of response, and improved quality of life and/or improving signs or symptoms of cancer.

As used herein, the term “progressive disease” (PD) refers to at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “partial response” (PR) refers to at least a 30% decrease in the sum of diameters of target lesions, taking as reference the baseline sum diameters.

As used herein, the term “complete response” (CR) refers to the disappearance of all target lesions with the short axes of any target lymph nodes reduced to <10 mm.

As used herein, the term “stable disease” (SD) refers to neither sufficient shrinkage to qualify for PR nor sufficient increase to qualify for PD, taking as reference the smallest sum of diameters while on study.

As used herein, the term “best overall response” (BOR) is the best response recorded from the start of the study treatment until the earliest of objective progression or start of new anti-cancer therapy, taking into account any requirement for confirmation. The patient's best overall response assignment will depend on the findings of both target and non-target disease and will also take into consideration the appearance of new lesions. The best overall response is calculated via an algorithm using the assessment responses provided by an investigator over the course of a trial.

As used herein, the term “not evaluable” (NE) refers to when an incomplete radiologic assessment of target lesions is performed or there is a change in the method of measurement from baseline that impacts the ability to make a reliable evaluation of response.

As used herein, the term “objective response rate” (ORR) is equal to the proportion of patients achieving a best overall response of partial or complete response (PR+CR) according to RECIST 1.1.

As used herein, the term “overall survival” (OS) refers to the percentage of patients remaining alive for a defined period of time, such as 1 year, 5 years, etc. from the time of diagnosis or treatment. Overall survival is evaluated by the Kaplan-Meier method, and a 95% confidence interval (CI) is provided for the median OS in each treatment arm.

As used herein, the term “progression-free survival” (PFS) refers to the patient remaining alive without the cancer progressing or getting worse. PFS may be defined as the time from selection for treatment until the first radiographic documentation of objective progression as defined by RECIST (Version 1.1), or death from any cause.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time, or where the administration of one therapeutic agent falls within a short period of time (e.g., within one day) relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes.

As used herein, “in conjunction with” refers to administration of one treatment modality in addition to another treatment modality. As such, “in conjunction with” refers to administration of one treatment modality before, during, or after administration of the other treatment modality to the individual.

The terms “label” and “detectable label” mean a moiety attached to a polynucleotide or polypeptide to render a reaction (for example, polynucleotide amplification or antibody binding) detectable. The polynucleotide or polypeptide comprising the label may be referred to as “detectably labeled.” Thus, the term “labeled binding protein” refers to a protein with a label incorporated that provides for the identification of the binding protein. The term “labeled oligonucleotide,” “labeled primer,” “labeled probe,” etc. refers to a polynucleotide with a label incorporated that provides for the identification of nucleic acids that comprise or are hybridized to the labeled oligonucleotide, primer, or probe. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In some embodiments, the moiety itself may not be detectably labeled but may become detectable upon reaction with yet another moiety.

The term “conjugate” refers to an antibody that is chemically linked to a second chemical moiety, such as a therapeutic or cytotoxic agent. The term “agent” includes a chemical compound, a mixture of chemical compounds, a biological macromolecule, or an extract made from biological materials. In some embodiments, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

As used herein, the term “flow cytometry” generally refers to a technique for characterizing biological particles, such as whole cells or cellular constituents, by flow cytometry. Methods for performing flow cytometry on samples of immune cells are well known in the art (see e.g., Jaroszeski et al., Method in Molecular Biology, (1998), vol. 91: Flow Cytometry Protocols, Humana Press; Longobanti Givan, (1992) Flow Cytometry, First Principles, Wiley Liss). All known forms of flow cytometry are intended to be included, particularly fluorescence activated cell sorting (FACS), in which fluorescent labeled molecules are evaluated by flow cytometry.

The term “amplification” refers to the process of producing one or more copies of a nucleic acid sequence or its complement. Amplification may be linear or exponential (e.g., PCR).

The technique of “polymerase chain reaction” or “PCR” as used herein generally refers to a procedure wherein a specific region of nucleic acid, such as RNA and/or DNA, is amplified as described, for example, in U.S. Pat. No. 4,683,195. Generally, oligonucleotide primers are designed to hybridize to opposite strands of the template to be amplified, a desired distance apart. PCR can be used to amplify specific RNA sequences, specific DNA sequences from total genomic DNA, and cDNA transcribed from total cellular RNA, bacteriophage or plasmid sequences, etc.

“Quantitative real time PCR” or “qRT-PCR” refers to a form of PCR wherein the PCR is performed such that the amounts, or relative amounts of the amplified product can be quantified. This technique has been described in various publications including Cronin et al., Am. J. Pathol. 164(I):35-42 (2004); and Ma et al., Cancer Cell 5:607-616 (2004).

The term “target sequence,” “target nucleic acid,” or “target nucleic acid sequence” refers generally to a polynucleotide sequence of interest, e.g., a polynucleotide sequence that is targeted for amplification using, for example, qRT-PCR.

The term “detection” includes any means of detecting, including direct and indirect detection.

In some embodiments, the terms “elevated levels of ICOS,” “elevated ICOS levels,” “ICOS at an elevated level,” “ICOS^{HIGH},” and “ICOS^{hi}” refer to increased levels of ICOS in cells (e.g., CD4+ T cells) of a subject, e.g., in a peripheral blood sample of the subject, after treatment of the subject with one or more anti-cancer therapies. The increased levels can be determined relative to a control, which may be, e.g., a peripheral blood sample from the subject being treated, but either before any treatment with the one or more anti-cancer therapies, or before treatment with a second or further cycle of the one or more anti-cancer therapies. Alternatively, the control can be a level from a matched sample (e.g., a peripheral blood sample) of a healthy individual. In some embodiments, the level of ICOS is determined at the level of expressed protein, which may be detected in some embodiments using an antibody directed to an intracellular portion of ICOS. In some embodiments, the detection using such an antibody is done by use of flow cytometry. In some embodiments, an increase of at least 2-fold (e.g., at least 3-fold, 4-fold, 5-fold, 7.5-fold, 10-fold, or 15-fold) in mean fluorescence intensity (MFI), relative to a control, indicates detection of elevated ICOS levels. In some embodiments, detection of an increase in ICOS levels in at least 5% (e.g., at least 10%, 20%, 30%, 40%, 50%, 60%, 70%, 80%, or 90%) of CD4+ T cells in a peripheral blood sample indicates a subject having an ICOS^{hi} sample. In some embodiments, an increase of at least 2-fold (e.g., at least 3-fold, 4-fold, 5-fold, 7.5-fold, 10-fold, or 15-fold) in mean fluorescence intensity (MFI), relative to a control, in at least 5% (e.g., at least 10%, 20%, 30%, 40%, 50%, 60%, 70%, 80%, or 90%) of CD4+ T cells in a peripheral blood sample indicates detection of elevated ICOS levels. In some embodiments, elevated ICOS levels refer to an increase in total ICOS expression levels (e.g., mRNA levels or protein levels) in CD4+ T cells in the peripheral blood test sample of about 10%, 20%, 30%, 40%, 50%, 60%, 70%, 80%, 85%, 90%, 95%, 100%, or greater relative to a control sample. In some embodiments, elevated ICOS levels refers to an increase in total ICOS expression levels (e.g., mRNA levels or protein levels) in the CD4+ T cells in a peripheral blood sample of about at least 1.1×, 2×, 3×, 4×, 5×, 10×, 15×, 20×, 30×, 40×, 50×, 100×, 500×, 1000×, or greater relative to a control sample.

Clonality can be a measure equal to the inverse of the normalized Shannon entropy of all productive clones in the sample. Primary measure of entropy is calculated by summing the frequency of each clone times the log (base 2) of the same frequency over all productive reads in a sample. When this value is normalized based on the total number of productive unique sequences and subtracted from 1, a related measure, ‘clonality’, results. In some embodiments, the clonality of TCR cells may be assessed by samples obtained from circulating tumor cells (CTCs), or circulating tumor DNA (ctDNA) in whole blood, serum, plasma, peripheral blood mononuclear cells (PBMCs) (“peripheral TCR clonality”).

### II. TCR Clonality and Determination of TCR clonality

T-lymphocytes respond to peptide fragments of protein antigens that are displayed by antigen presenting cells and MHC molecules. The receptor that recognizes these peptide-MHC complexes is the T-cell receptor (TCR). TCR may include antigen/MHC binding heterodimeric protein product of a vertebrate, e.g. mammalian, TCR gene complex, including the human TCR α, β, γ and δ chains. The TCR β-chain gene complex includes at least 57 variable (V) gene segments, which group into 24 TCR Vβ gene families. These TCR gene families are defined as a TCR repertoire. At a molecular level, the TCR is a heterodimer consisting of an α chain and a β chain.

Structurally, each chain has a variable region (V region), which allows binding to diverse peptide antigens, and a constant region (C region). Extensive variations at the V region are generated through somatic recombination of variable (V), diversity (D), and joining (J) gene segments of the TCR a and β chains during T-cell development. The V region of the β chain is the most polymorphic, and gives rise to the most diversity. In humans, there are 54 V genes and 13 J genes, and any one of the V genes can pair with any one of 13 J genes to generate an extremely diverse TCR repertoire. Within the variable region of each TCR, there are 3 Complementarity-Determining Regions (CDR), and the CDR3 region is in direct contact with peptide antigens that are presented by the MHC-peptide complex and responsible for antigen binding, and therefore, CDR3 gives rise to the highest degree of diversity (Robins HS, et al. Blood 2009 114(19):4099-4107).

Clonality can be a measure equal to the inverse of the normalized Shannon entropy of all productive clones in the sample. Primary measure of entropy is calculated by summing the frequency of each clone times the log (base 2) of the same frequency over all productive reads in a sample. When this value is normalized based on the total number of productive unique sequences and subtracted from 1, a related measure, ‘clonality’, results.

Values for clonality range from 0 to 1. Values near 1 represent samples with one or a few predominant clones (monoclonal or oligoclonal samples) dominating the observed repertoire. Clonality values near 0 represent more polyclonal samples. In some embodiments, a clonality of 0.50 or less may be used to indicate a lower clonality (i.e., high diversity) of TCR repertoire. In some embodiments, a clonality of 0.50 or less may be used to indicate a lower clonality of TCR repertoire. In some embodiments, a clonality of 0.20 or less may be used to indicate a lower clonality of TCR repertoire. In some embodiments, a clonality of 0.09 or less may be used to indicate a lower clonality of TCR repertoire. In some embodiments, a clonality of 0.05 or less may be used to indicate a lower clonality of TCR repertoire. In some embodiments, a clonality of 0.01 or less may be used to indicate a lower clonality of TCR repertoire. In some embodiments, a clonality of 0.50 or less may be used to indicate a lower clonality of TCR repertoire, e.g., 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less, or a range bounded by any two of the above clonality values (e.g. 0.01 to 0.05, 0.04 to 0.1, or 0.09 to 0.20).

In some embodiments, the TCR clonality of a subject can be compared to the TCR clonality of a single healthy individual or the average TCR clonality of a population of healthy individuals determined by the same methods used to determine the subject's TCR clonality. The healthy individual or population of healthy individuals can share one or more factors with the subject chosen from age, gender, race, geographic location, socioeconomic status, history of alcohol consumption, and history of drug use. The average T cell clonality of a population of healthy individuals is known to be 0.01 to 0.24, for example, 0.06 or 0.07 in average for healthy population (N=421) as described in Emerson, Ryan O., et al. “Immunosequencing identifies signatures of cytomegalovirus exposure history and HLA-mediated effects on the T cell repertoire,” Nature genetics 49:5, 659 (2017). In some embodiments, the subject is designated as having a low TCR clonality when their TCR clonality is within 5% of the average T cell clonality of a population of healthy individuals. In some embodiments, the subject is designated as having a low TCR clonality when their T cell clonality is lower than the T cell clonality of average T cell clonality of a population of healthy individuals. In some embodiments, the subject is designated as having a low TCR clonality when their T cell clonality is lower by 0.30, 0.20,0.10, or 0.05 than the T cell clonality of average T cell clonality of a population of healthy individuals.

In some embodiments, the clonality of TCR cells may be assessed by samples obtained from circulating tumor cells (CTCs), or circulating tumor DNA (ctDNA) in whole blood, serum, plasma, peripheral blood mononuclear cells (PBMCs) (“peripheral TCR clonality”).

Tissue samples for nucleic acid extraction may be obtained from tumor, circulating tumor cells (CTCs), or circulating tumor DNA (ctDNA) in whole blood, serum, plasma, peripheral blood mononuclear cells (PBMCs), urine, draining lymph node (LN), cerebrospinal fluid (CSF). In some embodiments, the nucleic acid is isolated from the peripheral blood of the subject. In some embodiments, nucleic acid is isolated from the circulating tumor cells (CTCs). In some embodiments, circulating tumor DNA (ctDNA) is isolated from the peripheral blood of the subject.

In some embodiments, the methods provided herein include detecting TCR clonality at a nucleic acid (DNA or RNA) level. In some embodiments, TCR clonality is detected by sequencing a nucleic acid at all or part of the region encoding the TCR. In some embodiments, the methods of detection include isolating cells from a tumor or other appropriate tissue associated with the cancer in the subject and further isolating nucleic acid from the cells, and then analyzing the nucleic acid for the presence of the TCR clonality.

In some embodiments, the method of detecting includes sequencing all or a portion of the region of the DNA encoding TCR protein in one or more of the cells. In some embodiments, all or a portion of the mRNA encoding the TCR protein is sequenced. In some embodiments, the method of detecting includes sequencing all or a portion of the region of the DNA encoding TCR protein in one or more of the cells. In some embodiments, all or a portion of the mRNA encoding the TCR protein is sequenced. Any suitable methods known in the art to sequence the DNA or mRNA may be used.

In some embodiments, the region of the TCR to be sequenced comprises a beta chain. In some embodiments, the region of the TCR to be sequenced comprises a variable region or a joining region. In some embodiments, the region of the TCR to be sequenced comprises a variable region of a beta chain.

### Exemplary Nucleic Acid-Based Detection Methods

The diversity of the CDR3 region makes each CDR3 nucleotide sequence unique in individual T-cell clones. Based on the V-J usage in the CDR3 region, DEEP sequencing (NextGen DEEP sequencing) allows sequencing of the CDR3 region of the β chain in the entire TCR repertoire, thus allowing identification of individual T-cell clones and repertoire diversity in any given individual (Miconnet I. Curr Opin HIV AIDS 2012 7(I):64-70). It should be noted that the composition and identity of individual T-cell clones vary considerably among individuals in the general population due to differences in vaccination history, frequency and nature of infections, history of immune activation, and age, etc.

In some embodiments, the method uses IMMUNOSEQ™ technology (Adaptive Biotechnologies), which allows ultra-DEEP sequencing of the TCRb CDR3 region and reveals the clonal composition of T cell populations. Briefly, the basic principle is a multiplexed PCR method that amplifies all possible rearranged genomic TCR β sequences in any given individual using 52 forward primers, each specific to a specific TCR vβ segment, and 13 reverse primers, each specific to a specific TCR Iβ segment. High throughput reads of 60-bp length can be obtained using the Illumina HiSeq® System. The raw HiSeq® sequences can be processed to generate private and shard sequence database.

III. Therapeutic Methods

The present disclosure provides methods of treating cancer in subjects in need of such treatment. The methods include administering an effective amount of anti-ICOS agonist antibody to the subject, wherein the subject exhibits a peripheral T-cell receptor (TCR) clonality of 0.50 or less. In some embodiments, the method comprises (i) detecting that the subject exhibits a peripheral T-cell receptor (TCR) clonality of 0.50 or less, and (ii) following step (i), administering an effective amount of anti-ICOS agonist antibody to the subject. In various embodiments, the method comprises (i) determining whether the subject exhibits a peripheral T-cell receptor (TCR) clonality of 0.50 or less, and (ii) following step (i), if the subject exhibits the peripheral TCR clonality of 0.50 or less, administering an effective amount of anti-ICOS agonist antibody to the subject. In various embodiments, the method comprises (i) measuring the level of peripheral T-cell receptor (TCR) clonality, and (ii) following step (i), if the subject has a peripheral TCR clonality of 0.50 or less, administering an effective amount of anti-ICOS agonist antibody to the subject. In some embodiments, (ii) administering an effective amount of anti-ICOS agonist antibody to the subject is performed when the subject is determined to exhibit a peripheral TCR clonality of 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

In various embodiments, the method comprises (i) providing a subject having cancer previously determined to have a peripheral T-cell receptor (TCR) clonality of 0.50 or less, and (ii) administering an effective amount of anti-ICOS agonist antibody to the subject. In some embodiments, the subject is previously determined to have peripheral TCR clonality of 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

In various embodiments, the method comprises administering an effective amount of anti-ICOS agonist antibody to the subject, wherein the subject exhibits a peripheral T-cell receptor (TCR) clonality of 0.50 or less. In some embodiments, the subject exhibits a peripheral TCR clonality of 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

In various embodiments, the method of treating cancer in a subject in need thereof comprises administering an anti-ICOS agonist antibody selectively to a subject having a low TCR clonality. In some embodiments, the method includes (i) detecting that the cancer exhibits a low TCR clonality, and (ii) following step (i), administering an effective amount of anti-ICOS agonist antibody to the subject. In some embodiments, the method includes (i) determining whether the cancer exhibits a low TCR clonality, and (ii) following step (i), if the cancer has low TCR clonality, administering an effective amount of anti-ICOS agonist antibody to the subject. In some embodiments, the method includes (i) providing a subject having cancer previously determined to have a low TCR clonality, and (ii) administering an effective amount of anti-ICOS agonist antibody to the subject. In some embodiments, the method includes administering an effective amount of anti-ICOS agonist antibody to the subject, wherein the cancer exhibits a low TCR clonality. Optionally, the method further includes administering an additional therapeutic agent with the anti-ICOS agonist antibody, such as an immunotherapy agent, a chemotherapy agent, or an anti-CTLA4, anti-PD-1, or anti-PD-L1 antagonist antibody, and/or administering radiation therapy.

Patients that can be treated as described herein are patients having a cancer. The type of cancer can be any type of cancer listed herein or otherwise known in the art. Exemplary types of cancer include, but are not limited to, gastric cancer, breast cancer (e.g., triple negative breast cancer (TNBC)), lung cancer (e.g., non-small cell lung cancer (NSCLC)), melanoma, renal cell carcinoma (RCC), bladder cancer, endometrial cancer, diffuse large B-cell lymphoma (DLBCL), Hodgkin's lymphoma, ovarian cancer, and head and neck squamous cell cancer (HNSCC). Also see the definition of cancer, above, for additional cancer types that can be treated according to the methods of the present disclosure.

Patients that can be treated as described herein include patients who have not previously received an anti-cancer therapy and patients who have received previous (e.g., 1, 2, 3, 4, 5, or more) doses or cycles of one or more (e.g., 1, 2, 3, 4, 5, or more) anti-cancer therapies.

Any of the anti-cancer therapies listed herein and others known in the art can be used in connection with the methods of the present disclosure. In some embodiments, the one or more anti-cancer therapies is two or more anti-cancer therapies. In some embodiments, the one or more anti-cancer therapies is three or more anti-cancer therapies. Specific, non-limiting examples of anti-cancer therapies that can be used in the present disclosure including, e.g., immunotherapies, chemotherapies, and cancer vaccines, among others, are provided below.

In some embodiments, the anti-ICOS agonist antibody is administered to the patient multiple times at regular intervals. These multiple administrations can also be referred to as administration cycles or therapy cycles. In some embodiments, the anti-ICOS agonist antibody is administered to the patient for more than two cycles, more than three cycles, more than four cycles, more than five cycles, more than ten cycles, more than fifteen cycles, or more than twenty cycles.

In some embodiments, the regular interval is a dosage every week, a dosage every two weeks, a dosage every three weeks, a dosage every four weeks, a dosage every five weeks, a dosage every six weeks, a dosage every seven weeks, a dosage every eight weeks, a dosage every nine weeks, a dosage every ten weeks, a dosage every eleven weeks, or a dosage every twelve weeks.

In some embodiments, the method further includes halting the administration of the anti-ICOS agonist antibody after a pre-determined number of administration cycles. The predetermined number of administration cycles may be four or more cycles (e.g., five or more cycles, six or more cycles, or seven or more cycles, eight or more cycles, nine or more cycles, or ten or more cycles). In some embodiments, the method further includes halting the administration of the anti-ICOS agonist antibody after the pre-determined number of administration cycles (e.g., four or more cycles) and, optionally, the patient is determined to have progressive disease by a routine method known in the art (e.g., progressive disease identified by radiographic progression per RECIST 1.1 criteria; see, e.g., the criteria listed above).

### IV. Exemplary Therapeutic Anti-ICOS Antibodies for Use

Therapeutic anti-ICOS antibodies that can be used in the present disclosure include, but are not limited to, humanized antibodies, chimeric antibodies, human antibodies, and antibodies comprising any of the heavy chain and/or light chain CDRs discussed herein. In some embodiments, the antibody is an isolated antibody. In some embodiments, the antibody is a monoclonal antibody.

In some embodiments, the anti-ICOS antibody is an anti-ICOS agonist antibody. See WO 2016/154177 and WO 2017/070423, which are each specifically incorporated herein by reference. In some embodiments, the therapeutic anti-ICOS agonist antibody includes at least one, two, there, four, five, or all six CDRs selected from (a) HCDR1 comprising the amino acid sequence of SEQ ID NO: 5; (b) HCDR2 comprising the amino acid sequence of SEQ ID NO: 6; (c) HCDR3 comprising the amino acid sequence of SEQ ID NO: 7; (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10. In various embodiments, one or more of the CDRs includes a substitution or deletion that does not destroy specific binding to ICOS. In some embodiments, one or more of the CDRs includes 1, 2, 3, or more substitutions, which may optionally comprise substitutions with conservative amino acids. In some embodiments, one or more of the CDRs includes 1, 2, 3, or more deletions.

In some embodiments, the therapeutic anti-ICOS antibody comprises six CDRs including (a) HCDR1 comprising the amino acid sequence of SEQ ID NO: 5; (b) HCDR2 comprising the amino acid sequence of SEQ ID NO: 6; (c) HCDR3 comprising the amino acid sequence of SEQ ID NO: 7; (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, a therapeutic anti-ICOS antibody comprises a heavy chain variable region and a light chain variable region. In some embodiments, a therapeutic anti-ICOS antibody comprises at least one heavy chain comprising a heavy chain variable region and at least a portion of a heavy chain constant region, and at least one light chain comprising a light chain variable region and at least a portion of a light chain constant region. In some embodiments, a therapeutic anti-ICOS antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some embodiments, the heavy chain is the region of the anti-ICOS antibody that comprises the three heavy chain CDRs. In some embodiments, the light chain is the region of the therapeutic anti-ICOS antibody that comprises the three light chain CDRs.

In some embodiments, the therapeutic anti-ICOS antibody comprises at least one, at least two, or all three VH CDR sequences selected from (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7.

In some embodiments, the therapeutic antibody comprises at least one, at least two, or all three VL CDR sequences selected from (a) LCDR1 comprising the amino acid sequence of SEQ ID NO: 8; (b) LCDR2 comprising the amino acid sequence of SEQ ID NO: 9; and (c) LCDR3 comprising the amino acid sequence of SEQ ID NO: 10.

In some embodiments, the therapeutic anti-ICOS antibody comprises (I) a VH domain comprising at least one, at least two, or all three VH CDR sequences selected from (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7; and (II) a VL domain comprising at least one, at least two, or all three VL CDR sequences selected from (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, a therapeutic anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 3. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted, and/or deleted in SEQ ID NO: 3. In some embodiments, substitutions, insertions, or deletions occur in regions outside the CDRs (that is, in the FRs). Optionally, the therapeutic anti-ICOS antibody comprises the VH sequence in SEQ ID NO: 3, including post-translational modifications of that sequence.

In some embodiments, the VH comprises: (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7.

In some embodiments, a therapeutic anti-ICOS antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 4. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted, and/or deleted in SEQ ID NO: 4. In some embodiments, the substitutions, insertions, or deletions occur in regions outside the CDRs (that is, in the FRs). Optionally, the therapeutic anti-ICOS antibody comprises the VL sequence in SEQ ID NO: 4, including post-translational modifications of that sequence.

In some embodiments, the VL comprises: (a) LCDR1 comprising the amino acid sequence of SEQ ID NO: 8; (b) LCDR2 comprising the amino acid sequence of SEQ ID NO: 9; and (c) LCDR3 comprising the amino acid sequence of SEQ ID NO: 10.

In some embodiments, a therapeutic anti-ICOS antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 3 and a light chain variable domain (VL) having at least 90%, 91%, 92%, 93%, 94%, 95%, 96%, 97%, 98%, 99%, or 100% sequence identity to the amino acid sequence of SEQ ID NO: 4. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-ICOS antibody comprising that sequence retains the ability to bind to ICOS. In some embodiments, a total of 1 to 10 amino acids have been substituted, inserted, and/or deleted in SEQ ID NO: 3. In some embodiments, a total of 1 to 10 amino acids have been substituted, inserted, and/or deleted in SEQ ID NO: 4. In some embodiments, substitutions, insertions, or deletions occur in regions outside the CDRs (that is, in the FRs). Optionally, the therapeutic anti-ICOS antibody comprises the VH sequence in SEQ ID NO: 3 and the VL sequence of SEQ ID NO: 4, including post-translational modifications of one or both sequence.

In some embodiments, the therapeutic anti-ICOS antibody comprises (I) a VH domain comprising: (a) HCDR1 comprising the amino acid sequence of SEQ ID NO: 5; (b) HCDR2 comprising the amino acid sequence of SEQ ID NO: 6; and (c) HCDR3 comprising the amino acid sequence of SEQ ID NO: 7; and (II) a VL domain comprising: (d) LCDR1 comprising the amino acid sequence of SEQ ID NO: 8; (e) LCDR2 comprising the amino acid sequence of SEQ ID NO: 9; and (f) LCDR3 comprising the amino acid sequence of SEQ ID NO: 10.

In some embodiments, a therapeutic anti-ICOS antibody comprises a VH as in any of the embodiments provided herein, and a VL as in any of the embodiments provided herein. In some embodiments, the antibody comprises the VH and VL sequences in SEQ ID NO: 3 and SEQ ID NO: 4, respectively, including post-translational modifications of those sequences.

In some embodiments, a therapeutic anti-ICOS antibody comprises a heavy chain comprising the amino acid sequence of SEQ ID NO: 1, or a variant thereof.

In some embodiments, a therapeutic anti-ICOS antibody comprises a light chain comprising the amino acid sequence of SEQ ID NO: 2, or a variant thereof.

In some embodiments, a therapeutic anti-ICOS antibody comprises a heavy chain comprising the amino acid sequence of SEQ ID NO: 1 and a light chain comprising the amino acid sequence of SEQ ID NO: 2, or variants thereof.

In some embodiments, the therapeutic anti-ICOS antibody comprises the six CDRs as described above and binds to ICOS. In some embodiments, the therapeutic anti-ICOS antibody comprises the six CDRs as described above, binds to ICOS and increases the number of Teff cells and/or activates Teff cells and/or decreases the number of Treg cells and/or increases the ratio of Teff cells to Treg cells in a mammal, such as a human. In some embodiments, the Treg cells are CD4+ FoxP3+ T cells. In some embodiments, the Teff cells are CD8+ T cells. In some embodiments, the Teff cells are CD4+ FoxP3− T cells and/or CD8+ T cells.

Exemplary therapeutic anti-ICOS antibodies include, but are not limited to, JTX-2011 (Jounce Therapeutics; US 2016/0304610; WO 2016/154177; WO 2017/070423) and BMS-986226 (Bristol-Myers Squibb).

In general, therapeutic anti-ICOS antibodies can be administered in an amount in the range of about 10 μg/kg body weight to about 100 mg/kg body weight per dose. In some embodiments, therapeutic anti-ICOS antibodies may be administered in an amount in the range of about 50 μg/kg body weight to about 5 mg/kg body weight per dose. In some embodiments, therapeutic anti-ICOS antibodies may be administered in an amount in the range of about 100 μg/kg body weight to about 10 mg/kg body weight per dose. In some embodiments, therapeutic anti-ICOS antibodies may be administered in an amount in the range of about 100 μg/kg body weight to about 20 mg/kg body weight per dose. In some embodiments, therapeutic anti-ICOS antibodies may be administered in an amount in the range of about 0.5 mg/kg body weight to about 20 mg/kg body weight per dose. In some embodiments, anti-ICOS antibodies may be administered in an amount in the range of about 0.05 mg/kg body weight to about 10 mg/kg body weight per dose. In some embodiments, anti-ICOS antibodies may be administered in an amount in the range of about 5 mg/kg body weight or lower, for example less than 4, less than 3, less than 2, or less than 1 mg/kg of the antibody. In specific examples, therapeutic anti-ICOS antibodies are administered at 0.1 mg/kg, 0.3 mg/kg, or 1.0 mg/kg, once every 3, 6, 9, or 12 weeks.

### V. Exemplary Additional Therapeutic Agents for Combined Therapy

As examples, any anti-cancer therapeutic agent listed herein or otherwise known in the art, can be used in combination with therapeutic anti-ICOS agonist antibodies in connection with the methods described herein. Exemplary anti-cancer therapeutic agents and combined therapies with therapeutic anti-ICOS agonist antibodies and additional anti-cancer therapeutic agents are further described below.

### a. Immunotherapies

In some embodiments, the one or more anti-cancer therapies is an immunotherapy. The interaction between cancer and the immune system is complex and multifaceted. See de Visser et al., Nat. Rev. Cancer (2006) 6:24-37. While many cancer patients appear to develop an anti-tumor immune response, cancers also develop strategies to evade immune detection and destruction. Recently, immunotherapy has been developed for the treatment and prevention of cancer and other disorders. Immunotherapy provides the advantage of cell specificity that other treatment modalities lack. As such, methods for enhancing the efficacy of immune based therapies can be clinically beneficial.

### i. Anti-CTLA-4 Antagonist Antibodies

In some embodiments, the one or more anti-cancer therapies is an anti-CTLA-4 antagonist antibody. An anti-CTLA-4 antagonist antibody refers to an agent capable of inhibiting the activity of cytotoxic T-lymphocyte-associated protein 4 (CTLA-4), thereby activating the immune system. The CTLA-4 antagonist may bind to CTLA-4 and reverse CTLA-4-mediated immunosuppression. A non-limiting exemplary anti-CTLA4 antibody is ipilimumab (YERVOY®, BMS), which may be administered according to methods known in the art, e.g., as approved by the US FDA. For example, ipilimumab may be administered in the amount of 3 mg/kg intravenously over 90 minutes every three weeks for a total of 4 doses (unresectable or metastatic melanoma); or at 10 mg/kg intravenously over 90 minutes every three weeks for a total of 4 doses, followed by 10 mg/kg every 12 weeks for up to 3 years or until documented recurrence or unacceptable toxicity (adjuvant melanoma).

### ii. OX40 Agonist Antibodies

In some embodiments, the one or more anti-cancer therapies is an agonist anti-OX40 antibody. An OX40 agonist antibody refers to an agent that induces the activity of OX40, thereby activating the immune system and enhancing anti-tumor activity. Non-limiting, exemplary agonist anti-OX40 antibodies are Medi6469, Medimmune, and MOXR0916/RG7888, Roche. These antibodies may be administered according to methods and in regimens determined to be appropriate by those of skill in the art.

### iii. PD-1 Therapies

In some embodiments, the one or more anti-cancer therapies is a PD-1 therapy. A PD-1 therapy encompasses any therapy that modulates PD-1 binding to PD-L1 and/or PD-L2. PD-1 therapies may, for example, directly interact with PD-1 and/or PD-L1. In some embodiments, a PD-1 therapy includes a molecule that directly binds to and/or influences the activity of PD-1. In some embodiments, a PD-1 therapy includes a molecule that directly binds to and/or influences the activity of PD-L1. Thus, an antibody that binds to PD-1 or PD-L1 and blocks the interaction of PD-1 to PD-L1 is a PD-1 therapeutic. When a desired subtype of PD-1 therapy is intended, it will be designated by the phrase “PD-1 specific” for a therapy involving a molecule that interacts directly with PD-1, or “PD-L1 specific” for a molecule that interacts directly with PD-L1, as appropriate. Unless designated otherwise, all disclosure contained herein regarding PD-1 therapy applies to PD-1 therapy generally, as well as PD-1 specific and/or PD-L1 specific therapies.

Non-limiting, exemplary PD-1 therapies include nivolumab (OPDIVO®, BMS-936558, MDX-1106, ONO-4538); pidilizumab, lambrolizumab/pembrolizumab (KEYTRUDA, MK-3475); BGB-A317, tislelizumab (BeiGene/Celgene); durvalumab (anti-PD-L1 antibody, MEDI-4736; AstraZeneca/Medimmune); RG-7446; avelumab (anti-PD-L1 antibody; MSB-0010718C; Pfizer); AMP-224; BMS-936559 (anti-PD-L1 antibody); AMP-514; MDX-1105; A B-011; anti-LAG-3/PD-1; spartalizumab (CoStim/Novartis); anti-PD-1 antibody (Kadmon Pharm.); anti-PD-1 antibody (Immunovo); anti-TEVI-3/PD-1 antibody (AnaptysBio); anti-PD-L1 antibody (CoStim/Novartis); RG7446/MPDL3280A (anti-PD-L1 antibody, Genentech/Roche); KD-033 (Kadmon Pharm.); AGEN-2034 (Agenus); STI-A1010; STI-A1110; TSR-042; atezolizumab (TECENTRIQ™); and other antibodies that are directed against programmed death-1 (PD-1) or programmed death ligand 1 (PD-L1).

PD-1 therapies are administered according to regimens that are known in the art, e.g., US FDA-approved regimens. In one example, nivolumab is administered as an intravenous infusion over 60 minutes in the amount of 240 mg every two weeks (unresectable or metastatic melanoma, adjuvant treatment for melanoma, non-small cell lung cancer (NSCLC), advanced renal cell carcinoma, locally advanced renal cell carcinoma, MSI-H or dMMR metastatic colorectal cancer, and hepatocellular carcinoma) or in the amount of 3 mg/kg every three weeks (classical Hodgkin lymphoma; recurrent or metastatic squamous cell carcinoma of the head and neck). In another example, pembrolizumab is administered by intravenous infusion over 30 minutes in the amount of 200 mg, once every three weeks. In another example, atezolizumab is administered by intravenous infusion over 60 minutes in the amount of 1200 mg every three weeks. In another example, avelumab is administered by intravenous infusion over 60 minutes in the amount of 10 mg/kg every two weeks. In another example, durvalumab is administered by intravenous infusion over 60 minutes in the amount of 10 mg/kg every two weeks.

### iv. TIGIT Antagonists

In some embodiments, the one or more anti-cancer therapies is TIGIT antagonist. A TIGIT antagonist refers to an agent capable of antagonizing or inhibiting the activity of T-cell immunoreceptor with Ig and ITIM domains (TIGIT), thereby reversing TIGIT-mediated immunosuppression. A non-limiting exemplary TIGIT antagonist is BMS-986207 (Bristol-Myers Squibb/Ono Pharmaceuticals). These agents may be administered according to methods and in regimens determined to be appropriate by those of skill in the art.

### v. IDO inhibitors

In some embodiments, the one or more anti-cancer therapies is an IDO inhibitor. An IDO inhibitor refers to an agent capable of inhibiting the activity of indoleamine 2,3-dioxygenase (IDO) and thereby reversing IDO-mediated immunosuppression. The IDO inhibitor may inhibit IDO and/or ID02 (INDOL1). An IDO inhibitor may be a reversible or irreversible IDO inhibitor. A reversible IDO inhibitor is a compound that reversibly inhibits IDO enzyme activity either at the catalytic site or at a non-catalytic site while an irreversible IDO inhibitor is a compound that irreversibly inhibits IDO enzyme activity by forming a covalent bond with the enzyme. Non-limiting exemplary IDO inhibitors are described, e.g., in US 2016/0060237; and US 2015/0352206. Non-limiting exemplary IDO inhibitors include Indoximod (New Link Genetics), INCB024360 (Incyte Corp), 1-methyl-D-tryptophan (New Link Genetics), and GDC-0919/navoximod (Genentech/New Link Genetics). These agents may be administered according to methods and in regimens determined to be appropriate by those of skill in the art.

### vi. RORγ Agonists

In some embodiments, the one or more anti-cancer therapies is a RORγ agonist. RORγ agonists refer to an agent capable of inducing the activity of retinoic acid-related orphan receptor gamma (RORγ), thereby decreasing immunosuppressive mechanisms. Non-limiting exemplary RORγ agonists include, but are not limited to, LYC-55716 (Lycera/Celgene) and INV-71 (Innovimmune). These agents may be administered according to methods and in regimens determined to be appropriate by those of skill in the art.

### b. Chemotherapies

In some embodiments, the one or more anti-cancer therapies is a chemotherapeutic agent. Exemplary chemotherapeutic agents that can be used include, but are not limited to, capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXA E® (protein-bound paclitaxel), pemetrexed, vinorelbine, vincristine, erlotinib, afatinib, gefitinib, crizotinib, dabrafenib, trametinib, vemurafenib, and cobimetanib. These agents may be administered according to methods and in regimens determined to be appropriate by those of skill in the art.

### c. Cancer Vaccines

In some embodiments, the one or more anti-cancer therapies is a cancer vaccine. Cancer vaccines have been investigated as a potential approach for antigen transfer and activation of dendritic cells. In particular, vaccination in combination with immunologic checkpoints or agonists for co-stimulatory pathways have shown evidence of overcoming tolerance and generating increased anti-tumor response. A range of cancer vaccines have been tested that employ different approaches to promoting an immune response against the tumor (see, e.g., Emens LA, Expert Opin Emerg Drugs 13(2): 295-308 (2008)). Approaches have been designed to enhance the response of B cells, T cells, or professional antigen-presenting cells against tumors. Exemplary types of cancer vaccines include, but are not limited to, peptide-based vaccines that employ targeting distinct tumor antigens, which may be delivered as peptides/proteins or as genetically-engineered DNA vectors, viruses, bacteria, or the like; and cell biology approaches, for example, for cancer vaccine development against less well-defined targets, including, but not limited to, vaccines developed from patient-derived dendritic cells, autologous tumor cells or tumor cell lysates, allogeneic tumor cells, and the like.

Exemplary cancer vaccines include, but are not limited to, dendritic cell vaccines, oncolytic viruses, tumor cell vaccines, etc. In some embodiments, such vaccines augment the anti-tumor response. Examples of cancer vaccines also include, but are not limited to, MAGE3 vaccine (e.g., for melanoma and bladder cancer), MUC1 vaccine (e.g., for breast cancer), EGFRv3 (such as Rindopepimut, e.g., for brain cancer, including glioblastoma multiforme), or ALVAC-CEA (e.g., for CEA+ cancers).

Non-limiting exemplary cancer vaccines also include Sipuleucel-T, which is derived from autologous peripheral-blood mononuclear cells (PBMCs) that include antigen-presenting cells (see, e.g., Kantoff PW et al., N Engl J Med 363:411-22 (2010)). In Sipuleucel-T generation, the patient's

PBMCs are activated ex vivo with PA2024, a recombinant fusion protein of prostatic acid phosphatase (a prostate antigen) and granulocyte-macrophage colony-stimulating factor (an immune-cell activator). Another approach to a candidate cancer vaccine is to generate an immune response against specific peptides mutated in tumor tissue, such as melanoma (see, e.g., Carreno et al., Science 348:6236, 2015). Such mutated peptides may, in some embodiments, be referred to as neoantigens. As a non-limiting example of the use of neoantigens in tumor vaccines, neoantigens in the tumor predicted to bind the major histocompatibility complex protein HLA-A*02:01 are identified for individual patients with a cancer, such as melanoma. Dendritic cells from the patient are matured ex vivo, then incubated with neoantigens. The activated dendritic cells are then administered to the patient. In some embodiments, following administration of the cancer vaccine, robust T-cell immunity against the neoantigen is detectable.

In some such embodiments, the cancer vaccine is developed using a neoantigen. In some embodiments, the cancer vaccine is a DNA vaccine. In some embodiments, the cancer vaccine is an engineered virus comprising a cancer antigen, such as PROSTVAC (rilimogene galvacirepvec/rilimogene glafolivec). In some embodiments, the cancer vaccine comprises engineered tumor cells, such as GVAX, which is a granulocyte-macrophage colony-stimulating factor (GM-CSF) gene-transfected tumor cell vaccine (see, e.g., Nemunaitis, Expert Rev. Vaccines 4:259-274, 2005).

The vaccines may be administered according to methods and in regimens determined to be appropriate by those of skill in the art.

### d. Additional Exemplary Anti-Cancer Therapies

Further non-limiting, exemplary anti-cancer therapies include Luspatercept (Acceleron Pharma/Celgene); Motolimod (Array BioPharma/Celgene/VentiRx Pharmaceuticals/Ligand); GI-6301 (Globelmmune/Celgene/NantWorks); GI-6200 (Globelmmune/Celgene/NantWorks); BLZ-945

(Celgene/Novartis); ARRY-382 (Array BioPharma/Celgene), or any of the anti-cancer therapies provided in Table 2. These agents may be administered according to methods and in regimens determined to be appropriate by those of skill in the art. In some embodiments, the one or more anti-cancer therapies includes surgery and/or radiation therapy. Accordingly, the anti-cancer therapies can optionally be utilized in the adjuvant or neoadjuvant setting.

### e. Combinations

In various examples, an anti-ICOS agonist antibody (e.g., an antibody described above, such as JTX-2011) is administered in combination with another immunotherapy (see, e.g., above). In one example, an anti-ICOS agonist antibody (e.g., an antibody described above, such as JTX-2011) is administered in combination with a PD-1 therapy (e.g., a PD-1 therapy listed above). Thus, the present disclosure includes, in various examples, administration of an anti-ICOS agonist antibody (e.g., JTX-2011) in combination with one or more of nivolumab, pidilizumab, lambrolizumab/pembrolizumab, BGB-A317, tislelizumab, durvalumab, RG-7446, avelumab, AMP-224, BMS-936559, AMP-514, MDX-1105, A B-011, anti-LAG-3/PD-1, spartalizumab (CoStim/Novartis);

anti-PD-1 antibody (Kadmon Pharm.); anti-PD-1 antibody (Immunovo); anti-TEVI-3/PD-1 antibody (AnaptysBio); anti-PD-L1 antibody (CoStim/Novartis); RG7446/MPDL3280A, KD-033 (Kadmon Pharm.); AGEN-2034 (Agenus), STI-A1010, STI-A1110, TSR-042, atezolizumab, and other antibodies that are directed against programmed death-1 (PD-1) or programmed death ligand 1 (PD-L1). In one specific example, JTX-2011 is administered with nivolumab.

Optionally, the combinations noted above further include one or more additional anti-cancer agents (e.g., immunotherapies). Accordingly, the combinations noted above can optionally include one or more of an anti-CTLA-4 antagonist antibody (e.g., ipilimumab), an anti-OX40 antibody (e.g., Medi6469), or MOXR0916/RG7888), a TIGIT antagonist (e.g., BMS-986207), an IDO inhibitor (e.g., indoximod, INCB024360, 1-methyl-D-tryptophan, or GDC-0919/navoximod), an RORγ agonist (e.g., LYC-55716 and INV-71), or a chemotherapeutic agent (see, e.g., above), or a cancer vaccine (see, e.g., above).

In other examples, a combination of the present disclosure includes an anti-ICOS agonist antibody (e.g., an antibody described above, such as JTX-2011) and one or more of an anti-CTLA-4 antagonist antibody (e.g., ipilimumab), an anti-OX40 antibody (e.g., Medi6469), or MOXR0916/RG7888), a TIGIT antagonist (e.g., BMS-986207), an IDO inhibitor (e.g., indoximod, INCB024360, 1-methyl-D-tryptophan, or GDC-0919/navoximod), an RORγ agonist (e.g., LYC-55716 and INV-71), or a chemotherapeutic agent (see, e.g., above), or a cancer vaccine (see, e.g., above).

In various examples, the components of a combination are administered according to dosing regimens described herein (e.g., US FDA-approved dosing regimens; see above), or using other regimens determined to be appropriate by those of skill in the art.

### VI. Pharmaceutical Compositions and Dosing

Compositions including one or more anti-ICOS agonist antibody are provided in formulations with a wide variety of pharmaceutically acceptable carriers, as determined to be appropriate by those of skill in the art (see, for example, Gennaro, Remington: The Science and Practice of Pharmacy with

Facts and Comparisons: Drugfacts Plus, 20^{th} ed. (2003); Ansel et al., Pharmaceutical Dosage Forms and Drug Delivery Systems, 7^{th} ed., Lippincott, Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

Anti-cancer therapies are administered in the practice of the methods of the present disclosure as is known in the art (e.g., according to FDA-approved regimens) or as indicated elsewhere herein (see, e.g., above). In some embodiments, anti-cancer therapies of the present disclosure are administered in amounts effective for treatment of cancer. The therapeutically effective amount is typically dependent on the weight of the subject being treated, his or her physical or health condition, the extensiveness of the condition to be treated, the age of the subject being treated, pharmaceutical formulation methods, and/or administration methods (e.g., administration time and administration route).

In some embodiments, anti-cancer therapies can be administered in vivo by various routes, including, but not limited to, intravenous, intra-arterial, parenteral, intratumoral, intraperitoneal or subcutaneous. The appropriate formulation and route of administration can be selected by those of skill in the art according to the intended application.

### VII. EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Low TCR Repertoire Clonality Predicts Responses to Treatment with JTX-2011

### Study Design

Pre-treatment PBMC samples from 14 patients having gastric (N=8), head and neck squamous cell carcinoma (N=1), non-small cell lung cancer (N=2), and triple negative breast cancer (N=3) were analyzed for changes in TCR sequences determined based on productive clonality. The efficacy of a combined therapy of anti-ICOS agonist antibody, JTX-2011(at 0.3 mg/kg q3w) and nivolumab (at 240 mg/kg q3w) in the 14 patients was assessed by detecting an emergence of ICOS^{h}' CD4+ T cell population.

Post-treatment PBMCs were obtained from patient whole blood samples through a density gradient separation using BD Vacutainer CPT Mononuclear Cell Preparation. Isolated PBMC samples were then frozen and stored at −80° C. until use in a flow cytometry application. At the time of analysis, PBMC sample tubes were thawed in a 37° C. water bath for approximately 2 minutes. Each sample was then transferred to a 15 mL conical tube with FACS buffer (1×PBS, 2% FBS, 0.01% sodium azide, 2 mM EDTA), and the cells enumerated. Staining was performed on 1×10⁶ PBMCs per sample. Following enumeration, PBMCs were centrifuged at 500×g for 3 minutes to obtain a cell pellet. Excess buffer was aspirated and the cell pellet was re-suspended in FACS buffer. The cell pellet re-suspension was divided equally into the wells of a 96-well round bottom plate, and each well was then Fc blocked using 5 μL of Fc block (Human TruStain FcX, BioLegend Cat # 422302) per 1×10⁵ PBMCs for 20 minutes at room temperature. Following blocking, the plate was centrifuged at 500×g for 3 minutes and excess buffer was removed.

Wells designated for the primary staining cocktail to assess total ICOS levels received 100 μL of master staining mix containing anti-human CD3 (clone: UCHT1), anti-human CD4 (clone: OKT4), and JTX-2011 Dylight 650. Wells designated for the isotype staining cocktail received 100 μL of master staining mix containing anti-human CD-3 (clone: UCHT1), anti-human CD4 (clone: OKT4), and anti-RSV Dylight 650. Staining cocktails were incubated at 4° C. for 30 minutes and then were centrifuged at 500×g for 3 minutes, two times with FACS buffer to wash. All wells were then fixed and permeabilized for 30 minutes (eBioscience FOXP3/Transcription Factor Staining Buffer Set ref. # 00-5523-00 Life Technologies). Following permeabilization, the plate was centrifuged at 500×g for 3 minutes and excess buffer was removed. Wells designated for the primary staining cocktail received 100 μL of master staining mix diluted in 1× permeabilization buffer (eBioscience FOXP3/Transcription Factor Staining Buffer Set ref. # 00-5523-00 Life Technologies) containing anti-T-bet (clone: 4610), streptavidin PE (BioLegend Cat 405204), and biotinylated M13 anti-ICOS detection antibody (Jounce Therapeutics), which recognizes an internal epitope of ICOS.

Wells designated for the isotype control staining cocktail received 100 μL of master staining mix prepared in lx permeabilization buffer (eBioscience FOXP3/Transcription Factor Staining Buffer Set ref. # 00-5523-00 Life Technologies) containing Streptavidin PE alone.

Staining cocktails were incubated at 4° C. for 30 minutes. The plate was then centrifuged at 500×g for 3 minutes, two times with lx permeabilization buffer to wash. The contents of the wells were then re-suspended in 150 μL of FACS buffer. Stained samples were immediately analyzed using a BD FACS Canto flow cytometer, with resulting data analyzed using FlowJo analysis software.

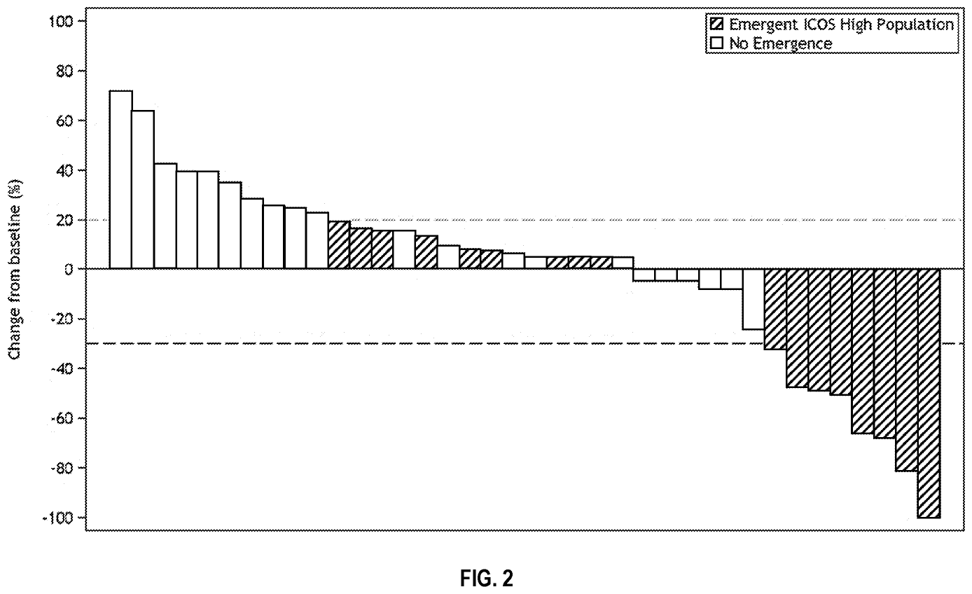

Correlation of the efficacy of anti-ICOS agonist antibody treatment with the emergence of ICOS^{h}'CD4+ T cell population has been discussed in U.S. Provisional Application No. 62/679,346. The emergence of this population correlated with evidence of biological activity corresponding to percent change from baseline of the target lesion size (See FIG. 2, adopted from U.S. Ser. No. 62/679,346), which supports the emergency of the ICOS^{hi} CD4+ T cell population (elevated ICOS levels) of as a surrogate marker for efficacy.

### Result

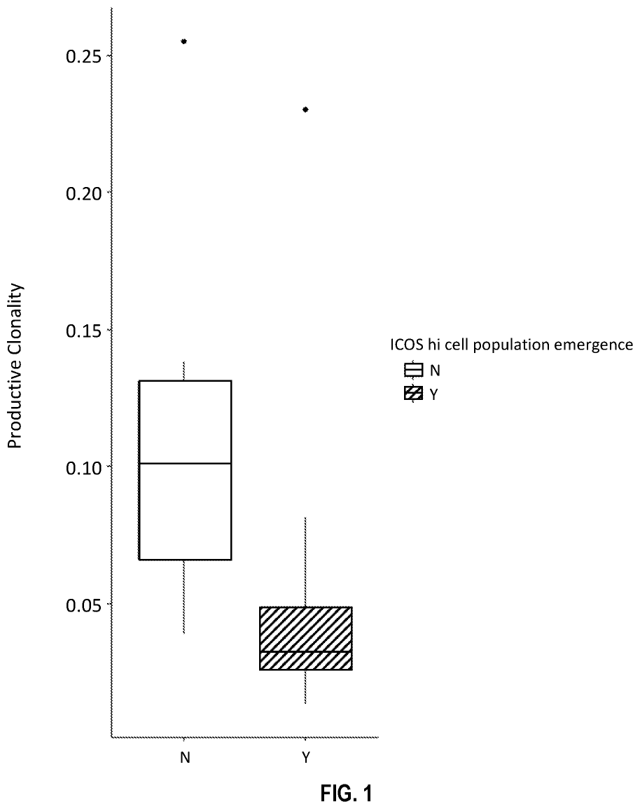

In FIG. 1, an emergence of ICOS^{hi} CD4+ T cell population was detected in the patients with the low peripheral TCR clonality with the median clonality value being less than 0.05. On the other hand, no emergence of ICOS^{hi} CD4+ T cell population was detected in the patients with the higher peripheral TCR clonality with the median clonality value being more than 0.10. It shows that the low peripheral TCR clonality in PBMC of patients prior to the therapy is associated with the emergence of the surrogate marker for efficacy. These data are consistent with the low clonality (i.e., high diversity) of TCR repertoire among patients responding to the above therapies.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

TABLE 2

| Anti-Cancer Therapeutic | Target Name |
|---|---|
| BMS-986179 | 5′-nucleotidase, ecto (CD73) |
| pTVG-HP | acid phosphatase, prostate |
| sipuleucel-T | acid phosphatase, prostate |
| CX-2009 | activated leukocyte cell adhesion molecule |
| luspatercept | activin A receptor type II-like 1 |
| CPI-444 | adenosine A2a receptor |
| NGR-TNF | alanyl (membrane) aminopeptidase |
| CB-1158 | arginase 1 |
| | arginase 2 |
| BA3011 | AXL receptor tyrosine kinase |
| AXL-107-MMAE | AXL receptor tyrosine kinase |
| CCT301-38 | AXL receptor tyrosine kinase |
| | RAR-related orphan receptor A |
| SurVaxM | baculoviral IAP repeat containing 5 |
| NY-ESO-1 TCR, | cancer/testis antigen 1 |
| Adaptimmune | |
| CDX-1401 | cancer/testis antigen 1 |
| | lymphocyte antigen 75 |
| ETBX-011 | carcinoembryonic antigen-related cell |
| | adhesion molecule 5 |
| GI-6207 | carcinoembryonic antigen-related cell |
| | adhesion molecule 5 |
| falimarev + inalimarev | carcinoembryonic antigen-related cell |
| | adhesion molecule 5 mucin 1, cell |
| | surface associated |
| labetuzumab govitecan | carcinoembryonic antigen-related cell |
| | adhesion molecule 5 topoisomerase |
| | (DNA) I |
| coltuximab ravtansine | CD19 molecule |
| denintuzumab mafodotin | CD19 molecule |
| axicabtagene ciloleucel | CD19 molecule |
| CIK-CAR.CD19 | CD19 molecule |
| JCAR014 | CD19 molecule |
| lisocabtagene maraleucel | CD19 molecule |
| tisagenlecleucel | CD19 molecule |
| MOR-208 | CD19 molecule |
| inebilizumab | CD19 molecule |
| AUTO3, Autolus | CD19 molecule |
| | CD22 molecule |
| DT2219ARL | CD19 molecule |
| | CD22 molecule |
| blinatumomab | CD19 molecule |
| | CD3e molecule, epsilon |
| | (CD3-TCR complex) |
| samalizumab | CD200 molecule |
| inotuzumab ozogamicin | CD22 molecule |
| 90Y-epratuzumab tetraxetan | CD22 molecule |
| epratuzumab | CD22 molecule |
| ontuxizumab | CD248 molecule, endosialin |
| varlilumab | CD27 molecule |
| durvalumab | CD274 molecule |
| avelumab | CD274 molecule |
| atezolizumab | CD274 molecule |
| CX-072 | CD274 molecule |
| enoblituzumab | CD276 molecule |
| omburtamab | CD276 molecule |
| AlloStim, Immunovative | CD28 molecule |
| Therapies | |
| gemtuzumab ozogamicin | CD33 molecule |
| lintuzumab-Ac225 | CD33 molecule |
| BI 836858 | CD33 molecule |
| naratuximab emtansine | CD37 molecule |
| lutetium (177Lu) | CD37 molecule |
| lilotomab satetraxetan | |
| otlertuzumab | CD37 molecule |
| daratumumab | CD38 molecule |
| isatuximab | CD38 molecule |
| TAK-573 | CD38 molecule |
| A-dmDT390-bisFv (UCHT1) | CD3e molecule, epsilon |
| | (CD3-TCR complex) |
| APX005M | CD40 molecule, TNF receptor |
| | superfamily member 5 |
| Hu5F9-G4 | CD47 molecule |
| TI-061 | CD47 molecule |
| milatuzumab | CD74 molecule, major histocompatibility |
| | complex, class II invariant chain |
| polatuzumab | CD79b molecule, immunoglobulin- |
| vedotin | associated beta |
| mogamulizumab | chemokine (C-C motif) receptor 4 |
| BL-8040 | chemokine (C-X-C motif) receptor 4 |
| X4P-001 | chemokine (C-X-C motif) receptor 4 |
| ulocuplumab | chemokine (C-X-C motif) receptor 4 |
| claudiximab | claudin 18 |
| ALT-836 | coagulation factor III (thromboplastin, |
| | tissue factor) |
| MCS110 | colony stimulating factor 1 (macrophage) |
| ARRY-382 | colony stimulating factor 1 (macrophage) |
| | colony stimulating factor 1 receptor |
| BLZ-945 | colony stimulating factor 1 receptor |
| AMG 820 | colony stimulating factor 1 receptor |
| cabiralizumab | colony stimulating factor 1 receptor |
| gemogenovatucel-T | colony stimulating factor 2 |
| | (granulocyte-macrophage) |
| GVAX | colony stimulating factor 2 |
| | (granulocyte-macrophage) |
| talimogene laherparepvec | colony stimulating factor 2 |
| | (granulocyte-macrophage) |
| pexastimogene devacirepvec | colony stimulating factor 2 |
| | (granulocyte-macrophage) |
| sargramostim | colony stimulating factor 2 |
| | receptor, alpha, low-affinity |
| | (granulocyte-macrophage) |
| SV-BR-1-GM cancer vaccine | colony stimulating factor 2 |
| | receptor, alpha, low-affinity |
| | (granulocyte-macrophage) |
| pamrevlumab | connective tissue growth factor |
| ipilimumab | cytotoxic T-lymphocyte-associated |
| | protein 4 |
| tremelimumab | cytotoxic T-lymphocyte-associated |
| | protein 4 |
| BMS-986249 | cytotoxic T-lymphocyte-associated |
| | protein 4 |
| rovalpituzumab tesirine | delta-like 3 (Drosophila) |
| ABT-165 | delta-like 4 (Drosophila) |
| | vascular endothelial growth factor A |
| BHQ880 | dickkopf WNT signaling pathway |
| | inhibitor 1 |
| DKN-01 | dickkopf WNT signaling pathway |
| | inhibitor 1 |
| Ad-REIC vaccine, | dickkopf WNT signaling pathway |
| Momotaro-Gene | inhibitor 3 |
| AGS-16C3F | ectonucleotide pyrophosphatase/ |
| | phosphodiesterase 3 |
| carotuximab | endoglin |
| ifabotuzumab | EPH receptor A3 |
| CimaVax EGF | epidermal growth factor |
| | (beta-urogastrone) |
| depatuxizumab mafodotin | epidermal growth factor receptor |
| RM-1929 | epidermal growth factor receptor |
| AVID100 | epidermal growth factor receptor |
| trastuzumab biosimilar, | epidermal growth factor receptor |
| Henlius | |
| cetuximab | epidermal growth factor receptor |
| panitumumab | epidermal growth factor receptor |
| necitumumab | epidermal growth factor receptor |
| nimotuzumab | epidermal growth factor receptor |
| futuximab | epidermal growth factor receptor |
| tomuzotuximab | epidermal growth factor receptor |
| doxorubicin, EDV nanocells, | epidermal growth factor receptor |
| EnGeneIC | |
| pan-HER | epidermal growth factor receptor |
| | erb-b2 receptor tyrosine kinase 2 |
| | erb-b2 receptor tyrosine kinase 3 |
| trastuzumab deruxtecan | erb-b2 receptor tyrosine kinase 2 |
| trastuzumab emtansine | erb-b2 receptor tyrosine kinase 2 |
| (vic-)trastuzumab | erb-b2 receptor tyrosine kinase 2 |
| duocarmazine | |
| nelipepimut-S | erb-b2 receptor tyrosine kinase 2 |
| trastuzumab biosimilar, | erb-b2 receptor tyrosine kinase 2 |
| Merck & Co./Samsung | |
| Bioepis | |
| trastuzumab biosimilar, | erb-b2 receptor tyrosine kinase 2 |
| Celltrion | |
| trastuzumab biosimilar, | erb-b2 receptor tyrosine kinase 2 |
| Biocon | |
| trastuzumab biosimilar, | erb-b2 receptor tyrosine kinase 2 |
| Allergan/Amgen | |
| trastuzumab biosimilar, | erb-b2 receptor tyrosine kinase 2 |
| Pfizer | |
| AU101, Aurora Biopharma | erb-b2 receptor tyrosine kinase 2 |
| AU105, Aurora BioPharma | erb-b2 receptor tyrosine kinase 2 |
| AE37 | erb-b2 receptor tyrosine kinase 2 |
| trastuzumab | erb-b2 receptor tyrosine kinase 2 |
| pertuzumab | erb-b2 receptor tyrosine kinase 2 |
| margetuximab | erb-b2 receptor tyrosine kinase 2 |
| ADXS31-164 | erb-b2 receptor tyrosine kinase 2 |
| ETBX-021 | erb-b2 receptor tyrosine kinase 2 |
| seribantumab | erb-b2 receptor tyrosine kinase 3 |
| patritumab | erb-b2 receptor tyrosine kinase 3 |
| CDX-3379 | erb-b2 receptor tyrosine kinase 3 |
| elgemtumab | erb-b2 receptor tyrosine kinase 3 |
| moxetumomab pasudotox | eukaryotic translation elongation factor 2 |
| | CD22 molecule |
| denileukin diftitox | eukaryotic translation elongation factor 2 |
| | interleukin 2 receptor, alpha |
| MDNA55 | eukaryotic translation elongation factor 2 |
| | interleukin 4 receptor |
| bemarituzumab | fibroblast growth factor receptor 2 |
| DCVax-prostate, Northwest | folate hydrolase (prostate-specific |
| Biotherapeutics | membrane antigen) 1 |
| 177Lu-J591 | folate hydrolase (prostate-specific |
| | membrane antigen) 1 |
| tuberculosis vaccine (Mw), | folate hydrolase (prostate-specific |
| Cadila; Cadi-05 | membrane antigen) 1 |
| mirvetuximab soravtansine | folate receptor 1 (adult) |
| TPIV200 | folate receptor 1 (adult) |
| farletuzumab | folate receptor 1 (adult) |
| IGEM-FR | folate receptor 1 (adult) |
| G17DT | gastrin |
| codrituzumab | glypican 3 |
| EP-100, EpiThany | gonadotropin-releasing hormone 1 |
| | (luteinizing-releasing hormone) |
| | luteinizing hormone/choriogonadotropin |
| | receptor |
| naxitamab | growth differentiation factor 2 |
| CDX-014 | hepatitis A virus cellular receptor 1 |
| MBG453 | hepatitis A virus cellular receptor 2 |
| histamine dihydrochloride | histamine receptor H2 |
| entinostat | histone deacetylase 1 |
| indoximod | indoleamine-pyrrole 2,3 dioxygenase |
| epacadostat | indoleamine-pyrrole 2,3 dioxygenase |
| BMS-986205 | indoleamine-pyrrole 2,3 dioxygenase |
| JTX-2011 | inducible T-cell co-stimulator |
| BMS-986226 | inducible T-cell co-stimulator |
| ADC W0101 | insulin-like growth factor 1 receptor |
| ganitumab | insulin-like growth factor 1 receptor |
| istiratumab | insulin-like growth factor 1 receptor |
| | erb-b2 receptor tyrosine kinase 3 |
| dusigitumab | insulin-like growth factor 1 receptor |
| | insulin-like growth factor 2 receptor |
| EP-201, EpiThany | insulin-like growth factor binding protein |
| | 2, 36kDa |
| citoplurikin | interferon gamma receptor 1 |
| | tumour necrosis factor receptor |
| | superfamily, member 1A |
| MABp1 | interleukin 1, alpha |
| pegilodecakin | interleukin 10 |
| Ad-RTS-hIL-12 + veledimex | interleukin 12 receptor, beta 1 |
| tavokinogene telsaplasmid | interleukin 12 receptor, beta 1 |
| | interleukin 12 receptor, beta 2 |
| EGEN-001 | interleukin 12A (natural killer cell |
| | stimulatory factor 1, cytotoxic |
| | lymphocyte maturation factor 1, p35) |
| | interleukin 12B (natural killer cell |
| | stimulatory factor 2, cytotoxic |
| | lymphocyte maturation factor 2, p40) |
| SL-701 | interleukin 13 receptor, alpha 2 |
| | EPH receptor A2 baculoviral IAP repeat |
| | containing 5 |
| ALT-803 | interleukin 15 receptor, alpha |
| Multikine, Cel-Sci | interleukin 2 receptor, alpha |
| ALT-801 | interleukin 2 receptor, alpha |
| high-affinity Natural Killer | interleukin 2 receptor, alpha |
| (haNK) cells, NantKwest | |
| interleukin-2, Roche | interleukin 2 receptor, alpha |
| aldesleukin | interleukin 2 receptor, alpha |
| NKTR-214 | interleukin 2 receptor, beta |
| talacotuzumab | interleukin 3 receptor, alpha |
| | (low affinity) |
| SL-401 | interleukin 3 receptor, alpha |
| | (low affinity) |
| siltuximab | interleukin 6 (interferon, beta 2) |
| HuMax-IL8 | interleukin 8 |
| PSA/IL-2/GM-CSF | kallikrein-related peptidase 3 |
| rilimogene galvacirepvec | kallikrein-related peptidase 3 |
| | CD80 molecule |
| | intercellular adhesion |
| | molecule 1 |
| | CD58 molecule |
| monalizumab | killer cell lectin-like receptor subfamily C, |
| | member 1 |
| ramucirumab | kinase insert domain receptor |
| ubenimex | leucotriene A4 hydrolase |
| | leucotriene B4 receptor |
| IMP321 | lymphocyte-activation gene 3 |
| LAG525 | lymphocyte-activation gene 3 |
| relatlimab | lymphocyte-activation gene 3 |
| imalumab | macrophage migration inhibitory factor |
| | (glycosylation-inhibiting factor) |
| OSE-2101 | major histocompatibility complex, |
| | class I, A |
| andecaliximab | matrix metallopeptidase 9 (gelatinase B, |
| | 92kDa gelatinase, 92kDa type IV |
| | collagenase) |
| anti-MAGE-A3 TCR, Kite | melanoma antigen family A, 3 |
| Pharma | |
| KITE-718 | melanoma antigen family A, 3 |
| biropepimut-S | melanoma antigen family A, 3 |
| rituximab biosimilar, Pfizer | membrane-spanning 4-domains, |
| | subfamily A, member 1 |
| rituximab biosimilar, Dr. | membrane-spanning 4-domains, |
| Reddy's | subfamily A, member 1 |
| rituximab biosimilar, Sandoz | membrane-spanning 4-domains, |
| | subfamily A, member 1 |
| rituximab biosimilar, | membrane-spanning 4-domains, |
| Celltrion | subfamily A, member 1 |
| rituximab biosimilar, | membrane-spanning 4-domains, |
| Archigen Biotech | subfamily A, member 1 |
| rituximab biosimilar, | membrane-spanning 4-domains, |
| Innovent Biologics | subfamily A, member 1 |
| MB-106 | membrane-spanning 4-domains, |
| | subfamily A, member 1 |
| ibritumomab tiuxetan | membrane-spanning 4-domains, |
| | subfamily A, member 1 |
| rituximab | membrane-spanning 4-domains, |
| | subfamily A, member 1 |
| ublituximab | membrane-spanning 4-domains, |
| | subfamily A, member 1 |
| rituximab biosimilar, | membrane-spanning 4-domains, |
| Allergan/Amgen | subfamily A, member 1 |
| ofatumumab | membrane-spanning 4-domains, |
| | subfamily A, member 1 |
| ocaratuzumab | membrane-spanning 4-domains, |
| | subfamily A, member 1 |
| veltuzumab | membrane-spanning 4-domains, |
| | subfamily A, member 1 |
| obinutuzumab | membrane-spanning 4-domains, |
| | subfamily A, member 1 |
| rituximab and hyaluronidase | membrane-spanning 4-domains, |
| human | subfamily A, member 1 |
| anetumab ravtansine | mesothelin |
| amatuximab | mesothelin |
| emibetuzumab | met proto-oncogene |
| binimetinib | mitogen-activated protein kinase kinase 1 |
| | mitogen-activated protein kinase kinase 2 |
| SAR566658 | mucin 1, cell surface associated |
| Cvac, Prima Biomed | mucin 1, cell surface associated |
| TG4010 | mucin 1, cell surface associated |
| | interleukin 2 receptor, alpha |
| oregovomab | mucin 16, cell surface associated |
| methionine enkephalin | opioid growth factor receptor |
| based immunotherapy | |
| olaratumab | platelet-derived growth factor receptor, |
| | alpha polypeptide |
| enfortumab vedotin | poliovirus receptor-related 4 |
| ProstAtak, Advantagene | polymerase (DNA directed), alpha 1, |
| | catalytic subunit |
| PancAtak, Advantagene | polymerase (DNA directed), alpha 1, |
| | catalytic subunit |
| aglatimagene besadenovec | polymerase (DNA directed), alpha 1, |
| | catalytic subunit |
| IMC-gp100 | premelanosome protein |
| cemiplimab | programmed cell death 1 |
| AGEN2034 | programmed cell death 1 |
| nivolumab | programmed cell death 1 |
| pembrolizumab | programmed cell death 1 |
| spartalizumab | programmed cell death 1 |
| BGB-A317 | programmed cell death 1 |
| genolimzumab | programmed cell death 1 |
| JNJ-63723283 | programmed cell death 1 |
| MEDI0680 | programmed cell death 1 |
| thymalfasin | prothymosin, alpha |
| LYC-55716 | RAR-related orphan receptor C |
| cirmtuzumab | receptor tyrosine kinase-like orphan |
| | receptor 1 |
| VX15/2503 | sema domain, immunoglobulin domain |
| | (Ig), transmembrane domain (TM) and |
| | short cytoplasmic domain, (semaphorin) |
| | 4D |
| elotuzumab | SLAM family member 7 |
| indatuximab ravtansine | syndecan 1 |
| BMS-986207 | T-cell immunoreceptor with Ig and ITIM |
| | domains |
| tertomotide | telomerase reverse transcriptase |
| Toca 511 + Toca FC | thymidylate synthetase |
| APS001F | thymidylate synthetase |
| JCARH125 | TNF receptor superfamily member 17 |
| bb2121 | TNF receptor superfamily member 17 |
| AUTO2, Autolus | TNF receptor superfamily member 17 |
| | TNF receptor superfamily member 13B |
| OPN-305 | toll-like receptor 2 |
| rintatolimod | toll-like receptor 3 |
| poly-ICLC | toll-like receptor 3 |
| ID-G100 | toll-like receptor 4 |
| ID-CMB305 | toll-like receptor 4 cancer/testis antigen 1 |
| imiquimod (intravesical), | toll-like receptor 7 |
| Telormedix | |
| NKTR-262 | toll-like receptor 7 |
| | toll-like receptor 8 |
| motolimod | toll-like receptor 8 |
| tilsotolimod | toll-like receptor 9 |
| sacituzumab govitecan | topoisomerase (DNA) I tumor-associated |
| | calcium signal transducer 2 |
| HPV-16 E6 TCR, Bluebird | transforming protein E6, human |
| Bio/Kite Pharma | papilloma virus-16 |
| VGX-3100 | transforming protein E6, human |
| | papilloma virus-16 |
| | transforming protein E7, human |
| | papilloma virus-16 |
| | E6 protein, human papilloma virus-18 |
| | E7 protein, human papilloma virus-18 |
| MEDI0457 | transforming protein E6, human |
| | papilloma virus-16 |
| | transforming protein E7, human |
| | papilloma virus-16 |
| | E7 protein, human papilloma virus-18 |
| | E6 protein, human papilloma virus-18 |
| TVGV-1 | transforming protein E7, human |
| | papilloma virus-16 |
| KITE-439 | transforming protein E7, human |
| | papilloma virus-16 |
| ADXS-DUAL | transforming protein E7, human |
| | papilloma virus-16 |
| axalimogene filolisbac | transforming protein E7, human |
| | papilloma virus-16 |
| MVA-5T4 | trophoblast glycoprotein |
| oportuzumab monatox | tumor-associated calcium signal |
| | transducer 2 |
| denosumab | tumour necrosis factor (ligand) |
| | superfamily, member 11 |
| BION-1301 | tumour necrosis factor (ligand) |
| | superfamily, member 13 |
| belimumab | tumour necrosis factor (ligand) |
| | superfamily, member 13b |
| INCAGN1876 | tumour necrosis factor receptor |
| | superfamily, member 18 |
| BMS-986156 | tumour necrosis factor receptor |
| | superfamily, member 18 |
| INCAGN1949 | tumour necrosis factor receptor |
| | superfamily, member 4 |
| PF-04518600 | tumour necrosis factor receptor |
| | superfamily, member 4 |
| BMS-986178 | tumour necrosis factor receptor |
| | superfamily, member 4 |
| brentuximab vedotin | tumour necrosis factor receptor |
| | superfamily, member 8 |
| urelumab | tumour necrosis factor receptor |
| | superfamily, member 9 |
| utomilumab | tumour necrosis factor receptor |
| | superfamily, member 9 |
| VBI-1901 | UL83, cytomegalovirus |
| | UL55, cytomegalovirus |
| bevacizumab biosimilar, | vascular endothelial growth factor A |
| Boehringer Ingelheim | |
| bevacizumab-awwb | vascular endothelial growth factor A |
| bevacizumab biosimilar, | vascular endothelial growth factor A |
| Pfizer | |
| bevacizumab biosimilar, | vascular endothelial growth factor A |
| Oncobiologics | |
| bevacizumab biosimilar, | vascular endothelial growth factor A |
| Henlius Biopharmaceuticals | |
| bevacizumab biosimilar, | vascular endothelial growth factor A |
| Fujifilm Kyowa Kirin | |
| Biologics | |
| aflibercept | vascular endothelial growth factor A |
| bevacizumab | vascular endothelial growth factor A |
| pritumumab | vimentin |
| pexidartinib | v-kit Hardy-Zuckerman 4 feline sarcoma |
| | viral oncogene homologue |
| | colony stimulating factor 1 receptor |
| | fms-related tyrosine kinase 3 |
| galinpepimut-S | Wilms tumour 1 |
| adegramotide/nelatimotide | Wilms tumour 1 |
| JTCR016 | Wilms tumour 1 |
| levamisole | Unknown |
| ladiratuzumab vedotin | Unknown |
| NSC-631570 | Unknown |
| LN-145 | Unknown |
| INO-5401 | Unknown |
| AN01, Anson Pharma | Unknown |
| GALE-302 | Unknown |
| MAGE-A3 TCR, | Unknown |
| Adaptimmune | |
| BTH-1677 | Unknown |
| lentinan | Unknown |
| Polysaccharide-K | Unknown |
| Tice BCG, Organon | Unknown |
| IGEM-F | Unknown |
| PV-10, Provectus | Unknown |
| vitespen | Unknown |
| mifamurtide | Unknown |
| melanoma vaccine, GSK | Unknown |
| Bacille Calmette-Guerin | Unknown |
| vaccine, ID Biomedical | |
| seviprotimut-I | Unknown |
| in situ autologous cancer | Unknown |
| vaccine, Immunophotonics | |
| IMA901 | Unknown |
| adagloxad simolenin | Unknown |
| PVX-410 | Unknown |
| viagenpumatucel-L | Unknown |
| GALE-301 | Unknown |
| EP-302, EpiThany | Unknown |
| BI 1361849 | Unknown |
| DPV-001 | Unknown |
| Bacille Calmette-Guerin | Unknown |
| vaccine, Sanofi | |
| LAMP-Vax + pp65 DC, | Unknown |
| Immunomic Therapeutics | |
| NKG2D-CAR | Unknown |
| BPX-501 | Unknown |
| NK-92 cells | Unknown |
| LN-144 | Unknown |
| CLBS-23 | Unknown |
| DCVax-Direct, Northwest | Unknown |
| Biotherapeutics | |
| melanoma vaccine, AVAX | Unknown |
| stapuldencel-T | Unknown |
| dendritic cancer vaccine, | Unknown |
| DanDrit Biotech | |
| DCVax-Brain brain cancer | Unknown |
| vaccine, Northwest | |
| Biotherapeutics | |
| tumor lysate particle-loaded | Unknown |
| dendritic cell vaccine, | |
| Perseus | |
| ERC1671 | Unknown |
| BSK01 TAPA pulsed DC | Unknown |
| vaccine | |
| Oncoquest-CLL vaccine | Unknown |
| rocapuldencel-T | Unknown |
| ATIR-101 | Unknown |
| TVI-Kidney-1 | Unknown |
| TVAX cancer vaccine, | Unknown |
| TVAX Biomedical | |
| atezolizumab, companion | Unknown |
| diagnostic | |
| tumour infiltrating | Unknown |
| lymphocytes, | |
| Iovance Biotherapeutics-2 | |
| MAGE A-10 TCR, | Unknown |
| Adaptimmune | |
| IMA101 | Unknown |
| algenpantucel-L | Unknown |
| Tumor Necrosis Therapy, | Unknown |
| Peregrine | |
| imiquimod | Unknown |
| LOAd703 | Unknown |
| CG0070 | Unknown |
| dinutuximab | Unknown |
| bavituximab | Unknown |
| ensituximab | Unknown |
| pidilizumab | Unknown |
| BMS-986218 | Unknown |
| BMS-986012 | Unknown |
| ADXS31-142 | Unknown |
| GI-6301 | Unknown |
| GI-4000 | Unknown |
| JNJ-64041757 | Unknown |
| HPV vaccine (Cervarix), | Unknown |
| GSK | |
| HPV vaccine (Gardasil), | Unknown |
| CSL | |
| Sym015 | Unknown |
| diphenylcyclopropenone | Unknown |
| ISA101 | Unknown |

TABLE 3
Sequences

| Name | | | |
|---|---|---|---|
| (Target, if | | SEQ | |
| applicable) | Region | ID | Sequence |
| JTX-2011 | Heavy | 1 | EVQLVESGGGLVQPGGSLRLSCAASGFTFSDYWMDWVRQAPGKGLVWVSNI |
| (ICOS) | Chain | | DEDGSITEYSPFVKGRFTISRDNAKNTLYLQMNSLRAEDTAVYYCTRWGRF |
| | | | GFDSWGQGTLVTVSSASTKGPSVFPLAPSSKSTSGGTAALGCLVKDYFPEP |
| | | | VTVSWNSGALTSGVHTFPAVLQSSGLYSLSSVVTVPSSSLGTQTYICNVNH |
| | | | KPSNTKVDKKVEPKSCDKTHTCPPCPAPELLGGPSVFLFPPKPKDTLMISR |
| | | | TPEVTCVVVDVSHEDPEVKFNWYVDGVEVHNAKTKPREEQYNSTYRVVSVL |
| | | | TVLHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPG |
| JTX-2011 | Light | 2 | DIVMTQSPDSLAVSLGERATINCKSSQSLLSGSFNYLTWYQQKPGQ |
| (ICOS) | Chain | | PPKLLIFYASTRHTGVPDRFSGSGSGTDFTLTISSLQAEDVAVYYC |
| | | | HHHYNAPPTFGPGTKVDIKRTVAAPSVFIFPPSDEQLKSGTASVVC |
| | | | LLNNFYPREAKVQWKVDNALQSGNSQESVTEQDSKDSTYSLSSILT |
| | | | LSKADYEKHKVYACEVTHQGLSSPVTKSFNRGEC |
| JTX-2011 | Heavy | 3 | EVQLVESGGGLVQPGGSLRLSCAASGFTFSDYWMDWVRQAPGKGLVWVSNI |
| (ICOS) | Chain | | DEDGSITEYSPFVKGRFTISRDNAKNTLYLQMNSLRAEDTAVYYCTRWGRF |
| | Variable | | GFDSWGQGTLVTVSS |
| | Region | | |
| JTX-2011 | Light | 4 | DIVMTQSPDSLAVSLGERATINCKSSQSLLSGSFNYLTWYQQKPGQPPKLL |
| (ICOS) | Chain | | IFYASTRHTGVPDRFSGSGSGTDFTLTISSLQAEDVAVYYCHHHYNAPPTF |
| | Variable | | GPGTKVDIK |
| | Region | | |
| JTX-2011 | HCDR1 | 5 | GFTFSDYWMD |
| (ICOS) | | | |
| JTX-2011 | HCDR2 | 6 | NIDEDGSITEYSPFVK |
| (ICOS) | | | |
| JTX-2011 | HCDR3 | 7 | WGRFGFDS |
| (ICOS) | | | |
| JTX-2011 | LCDR1 | 8 | KSSQSLLSGSFNYLT |
| (ICOS) | | | |
| JTX-2011 | LCDR2 | 9 | YASTRHT |
| (ICOS) | | | |
| JTX-2011 | LCDR3 | 10 | HHHYNAPPT |
| (ICOS) | | | |
| Human ICOS | | 11 | MKSGLWYFFLFCLRIKVLTGEINGSANYEMFIFHNGGVQILCKYPDIVQQF |
| precursor | | | KMQLLKGGQILCDLTKTKGSGNTVSIKSLKFCHSQLSNNSVSFFLYNLDHS |
| with signal | | | HANYYFCNLSIFDPPPFKVTLTGGYLHIYESQLCCQLKFWLPIGCAAFVVV |
| sequence | | | CILGCILICWLTKKKYSSSVHDPNGEYMFMRAVNTAKKSRLTDVTL |
| | | | (Intra-cellar Region is under-lined) |
| Human ICOS, | | 12 | EINGSANYEMFIFHNGGVQILCKYPDIVQQFKMQLLKGGQILCDLTKTKGS |
| mature | | | GNTVSIKSLKFCHSQLSNNSVSFFLYNLDHSHANYYFCNLSIFDPPPFKVT |
| | | | LTGGYLHIYESQLCCQLKFWLPIGCAAFVVVCILGCILICWLTKKKYSSSV |
| | | | HDPNGEYMINTRAVNTAKKSRLTDVTL |
| | | | (Intra-cellar Region is under-lined) |
| Mouse (Mus | | 13 | MKPYFCRVFVFCFLIRLLTGEINGSADHRMFSFHNGGVQISCKYPE |
| musculus) | | | TVQQLKMRLFREREVLCELTKTKGSGNAVSIKNPMLCLYHLSNNSV |
| ICOS | | | SFFLNNPDSSQGSYYFCSLSIFDPPPFQ |
| precursor | | | ERNLSGGYLHIYESQLCCQLKLWLPVGCAAFVVVLLFGCILIIWFS |
| | | | KKKYGSSVHDPNSEYMFMAAVNTNKKSRLAGVTS |
| Mouse (Mus | | 14 | EINGSADHRMFSFHNGGVQISCKYPETVQQLKMRLFREREVLCELT |
| musculus) | | | KTKGSGNAVSIKNPMLCLYHLSNNSVSFFLNNPDSSQGSYYFCSLS |
| ICOS, mature | | | IFDPPPFQERNLSGGYLHIYESQLCCQLKLWLPVGCAAFVVVLLFG |
| | | | CILIIWFSKKKYGSSVHDPNSEYMFMAAVNTNKKSRLAGVTS |
| Rat (Rattus | | 15 | MKPYFSCVFVFCFLIKLLTGELNDLANHRMFSFHDGGVQISCNYPE |
| norvegicus) | | | TVQQLKMQLFKDREVLCDLTKTKGSGNTVSIKNPMSCPYQLSNNSV |
| ICOS | | | SFFLDNADSSQGSYFLCSLSIFDPPPFQEKNLSGGYLLIYESQLCC |
| precursor | | | QLKLWLPVGCAAFVAALLFGCIFIVWFAKKKYRSSVHDPNSEYMFM |
| | | | AAVNTNKKSRLAGMTS |
| Rat (Rattus | | 16 | ELNDLANHRMFSFHDGGVQISCNYPETVQQLKMQLFKDREVLCDLTKTKGS |
| norvegicus) | | | GNTVSIKNPMSCPYQLSNNSVSFFLDNADSSQGSYFLCSLSIFDPPPFQEK |
| ICOS, mature | | | NLSGGYLLIYESQLCCQLKLWLPVGCAAFVAALLFGCIFIVWFAKKKYRSS |
| | | | VHDPNSEYMFMAAVNTNKKSRLAGMTS |
| Cynomolgus | | 17 | MKSGLWYFFLFCLHMKVLTGEINGSANYEMFIFHNGGVQILCKYPDIVQQF |
| monkey | | | KMQLLKGGQILCDLTKTKGSGNKVSIKSLKFCHSQLSNNSVSFFLYNLDRS |
| (Macaca | | | HANYYFCNLSIFDPPPFKVTLTGGYLHIYESQLCCQLKFWLPIGCATFVVV |
| fascicularis) | | | CIFGCILICWLTKKKYSSTVHDPNGEYMFMRAVNTAKKSRLTGTTP |
| ICOS, | | | |
| precursor | | | |
| Cynomolgus | | 18 | EINGSANYEMFIFHNGGVQILCKYPDIVQQFKMQLLKGGQILCDLT |
| monkey | | | KTKGSGNKVSIKSLKFCHSQLSNNSVSFFLYNLDRSHANYYFCNLS |
| (Macaca | | | IFDPPPFKVTLTGGYLHIYESQLCCQLKFWLPIGCATFVVVCIFGC |
| fascicularis) | | | ILICWLTKKKYSSTVHDPNGEYMFMRAVNTAKKSRLTGTTP |
| ICOS, mature | | | |
| JNC-1 (PD-1) | Heavy | 19 | QVQLVQSGAEVKKPGASVKVSCKASGYTFPSYYMHWVRQAPGQGLEWMGII |
| | Chain | | NPEGGSTAYAQKFQGRVTMTRDTSTSTVYMELSSLRSEDTAVYYCARGGTY |
| | Variable | | YDYTYWGQGTLVTVSS |
| | Region | | |
| JNC-1 (PD-1) | HCDR1 | 20 | YTFPSYYMH |
| JNC-1 (PD-1) | HCDR2 | 21 | IINPEGGSTAYAQKFQG |
| JNC-1 (PD-1) | HCDR3 | 22 | ARGGTYYDYTY |
| JNC-1 (PD-1) | Light | 23 | DIQMTQSPSTLSASVGDRVTITCRASQSISSWLAWYQQKPGKAPKLLIYEA |
| | Chain | | SSLESGVPSRFSGSGSGTEFTLTISSLQPDDFATYYCQQYNSFPPTFGGGT |
| | Variable | | KVEIK |
| | Region | | |
| JNC-1 (PD-1) | LCDR1 | 24 | RASQSISSWLA |
| JNC-1 (PD-1) | LCDR2 | 25 | EASSLES |
| JNC-1 (PD-1) | LCDR3 | 26 | QQYNSFPPT |
| 2M13 (ICOS | Heavy | 27 | EVQLQQSGAELVRPGAVVKLSCKASGFDIKDYYMHWVQQRPEQGLEWIGWI |
| intra- | Chain | | DPENGNAVYDPQFQGKASITADTSSNTAYLQLSSLTSEDTAVYYCASDYYG |
| cellular) | Variable | | SKGYLDVWGAGTTVTVSS |
| | Region | | |
| 2M13 (ICOS | HCDR1 | 28 | DYYMH |
| intra- | | | |
| cellular) | | | |
| 2M13 (ICOS | HCDR2 | 29 | WIDPENGNAVYDPQFQG |
| intra- | | | |
| cellular) | | | |
| 2M13 (ICOS | HCDR3 | 30 | DYYGSKGYLDV |
| intra- | | | |
| cellular) | | | |
| 2M13 (ICOS | Light | 31 | QIVLTQSPTIMSASPGEKVTITCSASSSVSYMHWFQQKPGTSPKLWIYSTS |
| intra- | Chain | | NLASGVPARFGGSRSGTSYSLTISRMEAEDAATYYCQQRSSYPFTFGSGTK |
| cellular) | Variable | | LEIK |
| | Region | | |
| 2M13 (ICOS | LCDR1 | 32 | SASSSVSYMH |
| intra- | | | |
| cellular) | | | |
| 2M13 (ICOS | LCDR2 | 33 | STSNLAS |
| intra- | | | |
| cellular) | | | |
| 2M13 (ICOS | LCDR3 | 34 | QQRSSYPFT |
| intra- | | | |
| cellular) | | | |
| 2M19 (ICOS | Heavy | 35 | EVQLQQSGAELVRSGASVKLSCTTSAFNIIDYYMHWVIQRPEQGLEWIAWI |
| intra- | Chain | | DPENGDPEYAPKFQDKATMTTDTSSNTAYLQLSSLTSEDTAVYYCTAWRGF |
| cellular) | Variable | | AYWGQGTLVTVSA |
| | Region | | |
| 2M19 (ICOS | HCDR1 | 36 | DYYMH |
| intra- | | | |
| cellular) | | | |
| 2M19 (ICOS | HCDR2 | 37 | WIDPENGDPEYAPKFQD |
| intra- | | | |
| cellular) | | | |
| 2M19 (ICOS | HCDR3 | 38 | WRGFAY |
| intra- | | | |
| cellular) | | | |
| 2M19 (ICOS | Light | 39 | DVVMTQTPLSLPVSLGDQASISCRSSQSLVHSNGNTYLHWYLQKPGQSPKL |
| intra- | Chain | | LIYKVSNRFSGVPDRFSGSGSGTDFTLKISRVEAEDLGVYFCSQSIHVPPT |
| cellular) | Variable | | FGGGTKLEIK |
| | Region | | |
| 2M19 (ICOS | LCDR1 | 40 | RSSQSLVHSNGNTYLH |
| intra- | | | |
| cellular) | | | |
| 2M19 (ICOS | LCDR2 | 41 | KVSNRFS |
| intra- | | | |
| cellular) | | | |
| 2M19 (ICOS | LCDR3 | 42 | SQSIHVPPT |
| intra- | | | |
| cellular) | | | |

What is claimed is:

1. A method of treating cancer in a subject in need thereof, the method comprising:
(i) detecting that the subject exhibits a peripheral T-cell receptor (TCR) clonality of 0.50 or less, and
(ii) following step (i), administering an effective amount of anti-ICOS agonist antibody to the subject.

2. A method of treating cancer in a subject in need thereof, the method comprising:
(i) determining whether the subject exhibits a peripheral T-cell receptor (TCR) clonality of 0.50 or less, and
(ii) following step (i), if the subject exhibits the peripheral TCR clonality of 0.50 or less, administering an effective amount of anti-ICOS agonist antibody to the subject.

3. A method of treating cancer in a subject in need thereof, the method comprising:
(i) measuring the level of peripheral T-cell receptor (TCR) clonality, and
(ii) following step (i), if the subject has a peripheral TCR clonality of 0.50 or less, administering an effective amount of anti-ICOS agonist antibody to the subject.

4. A method of treating cancer in a subject in need thereof, the method comprising:
(i) providing a subject having cancer previously determined to have a peripheral T-cell receptor (TCR) clonality of 0.50 or less, and
(ii) administering an effective amount of anti-ICOS agonist antibody to the subject.

5. A method of treating cancer in a subject in need thereof, comprising administering an effective amount of anti-ICOS agonist antibody to the subject, wherein the subject exhibits a peripheral T-cell receptor (TCR) clonality of 0.50 or less.

6. The method of claims 1-3, wherein (ii) administering an effective amount of anti-ICOS agonist antibody to the subject is performed when the subject is determined to exhibit a peripheral TCR clonality of 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

7. The method of claim 4, wherein the subject is previously determined to have peripheral TCR clonality of 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

8. The method of claim 5, wherein the subject exhibits a peripheral TCR clonality of 0.40 or less, 0.30 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

9. The method of any one of claims 6-8, wherein the peripheral TCR clonality is 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, 0.01 or less.

10. The method of any one of claims 1-9, wherein detecting that the cancer exhibits a peripheral TCR clonality of 0.50 or less or determining whether the cancer exhibits a peripheral TCR clonality of 0.50 or less comprises measuring the level of peripheral TCR clonality.

11. The method of any one of claims 1-10, wherein measuring the level of peripheral TCR clonality comprises testing a sample from the subject.

12. The method of any one of claims 1-11, wherein measuring the level of peripheral TCR clonality comprises isolating peripheral cells from a tumor or other appropriate tissue associated with said cancer in said subject and testing the cells for presence of a peripheral TCR clonality of 0.5 or less.

13. The method of any one of claims 1-12, wherein measuring the level of peripheral TCR clonality comprises isolating peripheral blood mononuclear cells (PBMC) from a tumor or other appropriate tissue associated with said cancer in said subject and testing the cells for presence of peripheral TCR clonality of 0.5 or less.

14. The method of any one of claims 10-13, wherein measuring the level of peripheral TCR clonality comprises isolating nucleic acid from the peripheral blood of said subject and sequencing all or a portion of a region encoding T cell receptor protein.

15. The method of claim 14, wherein the region of the T cell receptor is a beta chain.

16. The method of claim 14, wherein the region of the T cell receptor is a variable region or a joining region.

17. The method of claim 14, wherein the region of the T cell receptor is a variable region of a beta chain.

18. The method of any one of claims 1-17, wherein the subject is a human subject.

19. The method of any one of claims 1-18, wherein the sample from the subject is peripheral blood mononuclear cells (PBMC).

20. The method of any one of claims 1-19, wherein the method further comprising (iii) after the administration, obtaining one or more peripheral blood test samples from the subject, (iii) measuring ICOS levels of CD4+ T cells present in the one or more peripheral blood test samples, (iv) determining if there is a population of CD4+ T cells having elevated ICOS in any of the one or more peripheral blood test samples when compared to a control, wherein detection of increased ICOS relative to a control indicates that the subject may benefit from the administration.

21. The method of any one of claims 1-19, wherein the anti-ICOS agonist antibody is chosen from JTX-2011, BMS-986226, and GSK3359609.

22. The method of claim 21, wherein the anti-ICOS agonist antibody is JTX-2011.

23. The method of any one of claims 1-19, wherein the anti-ICOS agonist antibody comprises a heavy chain and a light chain, and further comprises at least one CDR selected from the group consisting of: (a) an HCDR1 comprising the amino acid sequence of SEQ ID NO: 5; (b) an HCDR2 comprising the amino acid sequence of SEQ ID NO: 6; (c) an HCDR3 comprising the amino acid sequence of SEQ ID NO: 7; (d) an LCDR1 comprising the amino acid sequence of SEQ ID NO:
8; (e) an LCDR2 comprising the amino acid sequence of SEQ ID NO: 9; and (f) an LCDR3 comprising the amino acid sequence of SEQ ID NO: 10, wherein one or more of the CDRs comprises 1 or 2 amino acid substitutions.

24. The method of claim 23, wherein the anti-ICOS agonist antibody comprises (a) an HCDR1 comprising the amino acid sequence of SEQ ID NO: 5; (b) an HCDR2 comprising the amino acid sequence of SEQ ID NO: 6; (c) an HCDR3 comprising the amino acid sequence of SEQ ID NO:
7; (d) an LCDR1 comprising the amino acid sequence of SEQ ID NO: 8; (e) an LCDR2 comprising the amino acid sequence of SEQ ID NO: 9; and (f) an LCDR3 comprising the amino acid sequence of SEQ ID NO: 10.

25. The method of claim 23, wherein the anti-ICOS agonist antibody comprises (a) an HCDR1 comprising the amino acid sequence of SEQ ID NO: 5; (b) an HCDR2 comprising the amino acid sequence of SEQ ID NO: 6; (c) an HCDR3 comprising the amino acid sequence of SEQ ID NO: 7; (d) an LCDR1 comprising the amino acid sequence of SEQ ID NO: 8; (e) an LCDR2 comprising the amino acid sequence of SEQ ID NO: 9; and (f) an LCDR3 comprising the amino acid sequence of SEQ ID NO: 10.

26. The method of any one of claims 1-19, wherein the anti-ICOS agonist antibody comprises (a) a heavy chain variable domain (VH) sequence having at least 90%, 91%, 92%, 93%, 94%, 95%, 96%, 97%, 98%, 99%, or 100% sequence identity to the amino acid sequence of SEQ ID NO: 3; and/or (b) a light chain variable domain (VL) having at least 90%, 91%, 92%, 93%, 94%, 95%, 96%, 97%, 98%, 99%, or 100% sequence identity to the amino acid sequence of SEQ ID NO: 4.

27. The method of claim 26, wherein the anti-ICOS agonist antibody comprises (a) a heavy chain variable domain (VH) sequence comprising the amino acid sequence of SEQ ID NO: 3, and (b) a light chain variable domain (VL) comprising the amino acid sequence of SEQ ID NO: 4.

28. The method of any one of claims 1-19, wherein the anti-ICOS agonist antibody comprises (a) a heavy chain comprising the amino acid sequence of SEQ ID NO: 1 and/or (b) a light chain comprising the amino acid sequence of SEQ ID NO: 2.

29. The method of claim 28, wherein the anti-ICOS agonist antibody comprises (a) a heavy chain comprising the amino acid sequence of SEQ ID NO: 1 and (b) a light chain comprising the amino acid sequence of SEQ ID NO: 2

30. The method of any one of claims 1-29, wherein the anti-ICOS agonist antibody is administered at a dosage of from 0.1 mg/kg to 0.3 mg/kg.

31. The method of claim 30, wherein the anti-ICOS agonist antibody is administered at a dosage of 0.1 mg/kg, 0.2 mg/kg, or 0.3 mg/kg.

32. The method of claim 31, wherein the anti-ICOS agonist antibody is administered at a dosage of 0.3 mg/kg.

33. The method of any one of claims 1-32, wherein the anti-ICOS agonist antibody is administered at a frequency of weekly, once every two weeks, once every three weeks, once every four weeks, once every six weeks, once every nine weeks, or once every twelve weeks.

34. The method of any one of claims 1-33, wherein the method further comprises administering an additional therapeutic agent with the anti-ICOS agonist antibody.

35. The method of claim 34, wherein the additional therapeutic agent is an immunotherapeutic agent.

36. The method of claim 35, wherein the additional therapeutic agent is at least one of (i) an anti-CTLA-4 antagonist antibody, (ii) an anti-PD-1 or anti-PD-L1 antagonist antibody, and (iii) an agent listed in Table 2.

37. The method of claim 36, wherein the additional therapeutic agent comprises an anti-CTLA-4 antagonist antibody.

38. The method of claim 37, wherein the anti-CTLA-4 antagonist antibody is selected from ipilimumab, tremelimumab, and BMS-986249.

39. The method of claim 38, wherein the anti-CTLA-4 antagonist antibody is ipilimumab.

40. The method of any one of claims 34-39, wherein the additional therapeutic agent comprises an anti-PD-1 or anti-PD-L1 antagonist antibody.

41. The method of claim 40, wherein the anti-PD-1 or anti-PD-L1 antagonist antibody is chosen from avelumab, atezolizumab, CX-072, pembrolizumab, nivolumab, cemiplimab, spartalizumab, tislelizumab, JNJ-63723283, genolimzumab, AMP-514, AGEN2034, durvalumab, and JNC-1.

42. The method of claim 41, wherein the anti-PD-1 or anti-PD-L1 antagonist antibody is chosen from pembrolizumab, nivolumab, atezolizumab, avelumab, and duravalumab.

43. The method of any one of claims 34-42, wherein the additional therapeutic agent comprises one or more of the agents listed in Table 2.

44. The method of any one of claims 34-43, wherein the additional therapeutic agent further comprises a chemotherapy agent.

45. The method of claim 44, wherein the chemotherapy agent is selected from one or more of capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, pemetrexed, vinorelbine, vincristine, erlotinib, afatinib, gefitinib, crizotinib, dabrafenib, trametinib, vemurafenib, and cobimetanib.

46. The method of any one of claims 34-45, wherein the method further comprises administering radiation therapy.

47. The method of any one of claims 34-40, wherein the additional therapeutic agent is administered every week, every two weeks, every three weeks, every four weeks, every six weeks, every nine weeks, and every twelve weeks.

48. The method of any one of claims 1-47, wherein the cancer is selected from gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, and head and neck squamous cell cancer (HNSCC).

49. The method of claim 48, wherein the cancer is gastric cancer.

50. The method of claim 48, wherein the cancer is non-small cell lung cancer.

51. The method of any one of claims 48-50, wherein the cancer is metastasized to the ovary of said subject (i.e., a Krukenberg tumor).


SEQUENCE LISTING (43 sequences)

SEQ ID NO: 1 - protein, 446 aa, Artificial Sequence
  Synthetic JTX-2011 (ICOS), Heavy Chain
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 218 aa, Artificial Sequence
  Synthetic JTX-2011 (ICOS), Light Chain
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 117 aa, Artificial Sequence
  Synthetic JTX-2011 (ICOS), Heavy Chain Variable Region
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 4 - protein, 111 aa, Artificial Sequence
  Synthetic JTX-2011 (ICOS), Light Chain Variable Region
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 5 - protein, 10 aa, Artificial Sequence
  Synthetic JTX-2011 (ICOS), HCDR1
GFTFSDYWMD

SEQ ID NO: 6 - protein, 16 aa, Artificial Sequence
  Synthetic JTX-2011 (ICOS), HCDR2
NIDEDGSITEYSPFVK

SEQ ID NO: 7 - protein, 8 aa, Artificial Sequence
  Synthetic JTX-2011 (ICOS), HCDR3
WGRFGFDS

SEQ ID NO: 8 - protein, 15 aa, Artificial Sequence
  Synthetic JTX-2011 (ICOS), LCDR1
KSSQSLLSGSFNYLT

SEQ ID NO: 9 - protein, 7 aa, Artificial Sequence
  Synthetic JTX-2011 (ICOS), LCDR2
YASTRHT

SEQ ID NO: 10 - protein, 9 aa, Artificial Sequence
  Synthetic JTX-2011 (ICOS), LCDR3
HHHYNAPPT

SEQ ID NO: 11 - protein, 199 aa, Homo sapiens
  misc_feature (1)..(199): Human ICOS precursor with signal sequence
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 12 - protein, 179 aa, Homo sapiens
  misc_feature (1)..(179): Human ICOS, mature
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 13 - protein, 200 aa, Mus musculus
  misc_feature (1)..(200): Mouse(Mus musculus) ICOS precursor
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 14 - protein, 180 aa, Mus musculus
  misc_feature (1)..(180): Mouse (Mus musculus) ICOS, mature
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 15 - protein, 200 aa, Rattus norvegicus
  misc_feature (1)..(200): Rat (Rattus norvegicus) ICOS precursor
MKPYFSCVFVFCFLIKLLTGELNDLANHRMFSFHDGGVQISCNYPETVQQLKMQLFKDREVLCDLTKTKGSGNTVSIKNPMSCPYQLSNNSVSFFLDNADSSQGSYFLCSLSIFDPPPFQEKNLSGGYLLIYESQLCCQLKLWLPVGCAAFVAALLFGCIFIVWFAKKKYRSSVHDPNSEYMFMAAVNTNKKSRLAGMTS

SEQ ID NO: 16 - protein, 180 aa, Rattus norvegicus
  misc_feature (1)..(180): Rat (Rattus norvegicus) ICOS, mature
ELNDLANHRMFSFHDGGVQISCNYPETVQQLKMQLFKDREVLCDLTKTKGSGNTVSIKNPMSCPYQLSNNSVSFFLDNADSSQGSYFLCSLSIFDPPPFQEKNLSGGYLLIYESQLCCQLKLWLPVGCAAFVAALLFGCIFIVWFAKKKYRSSVHDPNSEYMFMAAVNTNKKSRLAGMTS

SEQ ID NO: 17 - protein, 199 aa, Macaca fascicularis
  misc_feature (1)..(199): Cynomolgus monkey (Macaca fascicularis) ICOS, precursor
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 18 - protein, 179 aa, Macaca fascicularis
  misc_feature (1)..(179): Cynomolgus monkey (Macaca fascicularis) ICOS, mature
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 19 - protein, 118 aa, Artificial Sequence
  Synthetic JNC-1 (PD-1), Heavy Chain Variable Region
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSS

SEQ ID NO: 20 - protein, 9 aa, Artificial Sequence
  Synthetic JNC-1 (PD-1), HCDR1
YTFPSYYMH

SEQ ID NO: 21 - protein, 17 aa, Artificial Sequence
  Synthetic JNC-1 (PD-1), HCDR2
IINPEGGSTAYAQKFQG

SEQ ID NO: 22 - protein, 11 aa, Artificial Sequence
  Synthetic JNC-1 (PD-1), HCDR3
ARGGTYYDYTY

SEQ ID NO: 23 - protein, 107 aa, Artificial Sequence
  Synthetic JNC-1 (PD-1), Light Chain Variable Region
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIK

SEQ ID NO: 24 - protein, 11 aa, Artificial Sequence
  Synthetic JNC-1 (PD-1), LCDR1
RASQSISSWLA

SEQ ID NO: 25 - protein, 7 aa, Artificial Sequence
  Synthetic JNC-1 (PD-1), LCDR2
EASSLES

SEQ ID NO: 26 - protein, 9 aa, Artificial Sequence
  Synthetic JNC-1 (PD-1), LCDR3
QQYNSFPPT

SEQ ID NO: 27 - protein, 120 aa, Artificial Sequence
  Synthetic 2M13 (ICOS intra-cellular), Heavy Chain Variable Region
EVQLQQSGAELVRPGAVVKLSCKASGFDIKDYYMHWVQQRPEQGLEWIGWIDPENGNAVYDPQFQGKASITADTSSNTAYLQLSSLTSEDTAVYYCASDYYGSKGYLDVWGAGTTVTVSS

SEQ ID NO: 28 - protein, 5 aa, Artificial Sequence
  Synthetic 2M13 (ICOS intra-cellular), HCDR1
DYYMH

SEQ ID NO: 29 - protein, 17 aa, Artificial Sequence
  Synthetic 2M13 (ICOS intra-cellular), HCDR2
WIDPENGNAVYDPQFQG

SEQ ID NO: 30 - protein, 11 aa, Artificial Sequence
  Synthetic 2M13 (ICOS intra-cellular), HCDR3
DYYGSKGYLDV

SEQ ID NO: 31 - protein, 106 aa, Artificial Sequence
  Synthetic 2M13 (ICOS intra-cellular), Light Chain Variable Region
QIVLTQSPTIMSASPGEKVTITCSASSSVSYMHWFQQKPGTSPKLWIYSTSNLASGVPARFGGSRSGTSYSLTISRMEAEDAATYYCQQRSSYPFTFGSGTKLEIK

SEQ ID NO: 32 - protein, 10 aa, Artificial Sequence
  Synthetic 2M13 (ICOS intra-cellular), LCDR1
SASSSVSYMH

SEQ ID NO: 33 - protein, 7 aa, Artificial Sequence
  Synthetic 2M13 (ICOS intra-cellular), LCDR2
STSNLAS

SEQ ID NO: 34 - protein, 9 aa, Artificial Sequence
  Synthetic 2M13 (ICOS intra-cellular), LCDR3
QQRSSYPFT

SEQ ID NO: 35 - protein, 115 aa, Artificial Sequence
  Synthetic 2M19 (ICOS intra-cellular), Heavy Chain Variable Region
EVQLQQSGAELVRSGASVKLSCTTSAFNIIDYYMHWVIQRPEQGLEWIAWIDPENGDPEYAPKFQDKATMTTDTSSNTAYLQLSSLTSEDTAVYYCTAWRGFAYWGQGTLVTVSA

SEQ ID NO: 36 - protein, 5 aa, Artificial Sequence
  Synthetic 2M19 (ICOS intra-cellular), HCDR1
DYYMH

SEQ ID NO: 37 - protein, 17 aa, Artificial Sequence
  Synthetic 2M19 (ICOS intra-cellular), HCDR2
WIDPENGDPEYAPKFQD

SEQ ID NO: 38 - protein, 6 aa, Artificial Sequence
  Synthetic 2M19 (ICOS intra-cellular), HCDR3
WRGFAY

SEQ ID NO: 39 - protein, 112 aa, Artificial Sequence
  Synthetic 2M19 (ICOS intra-cellular), Light Chain Variable Region
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSIHVPPTFGGGTKLEIK

SEQ ID NO: 40 - protein, 16 aa, Artificial Sequence
  Synthetic 2M19 (ICOS intra-cellular), LCDR1
RSSQSLVHSNGNTYLH

SEQ ID NO: 41 - protein, 7 aa, Artificial Sequence
  Synthetic 2M19 (ICOS intra-cellular), LCDR2
KVSNRFS

SEQ ID NO: 42 - protein, 9 aa, Artificial Sequence
  Synthetic 2M19 (ICOS intra-cellular), LCDR3
SQSIHVPPT

SEQ ID NO: 43 - protein, 188 aa, Homo sapiens [sequence omitted]